SEQUENCE LISTING (49 sequences)

SEQ ID NO: 1 - protein, 20 aa, Artificial Sequence
  synthetic peptide
LRRDLDAXXEAKXQVEXALE

SEQ ID NO: 2 - protein, 12 aa, Artificial Sequence
  synthetic peptide
XXEAKXQVEXAL

SEQ ID NO: 3 - protein, 12 aa, Artificial Sequence
  synthetic peptide
ENEAKKQVEKAL

SEQ ID NO: 4 - protein, 12 aa, Artificial Sequence
  synthetic peptide
EDEAKKQVEKAL

SEQ ID NO: 5 - protein, 12 aa, Artificial Sequence
  synthetic peptide
EREAKNQVEKAL

SEQ ID NO: 6 - protein, 12 aa, Artificial Sequence
  synthetic peptide
EREAKKQVERAL

SEQ ID NO: 7 - protein, 12 aa, Artificial Sequence
  synthetic peptide
EREAKKQVEMAL

SEQ ID NO: 8 - protein, 12 aa, Artificial Sequence
  synthetic peptide
VNEAKKQVEKAL

SEQ ID NO: 9 - protein, 12 aa, Artificial Sequence
  synthetic peptide
VDEAKKQVEKAL

SEQ ID NO: 10 - protein, 12 aa, Artificial Sequence
  synthetic peptide
VREAKNQVEKAL

SEQ ID NO: 11 - protein, 12 aa, Artificial Sequence
  synthetic peptide
VREAKKQVERAL

SEQ ID NO: 12 - protein, 12 aa, Artificial Sequence
  synthetic peptide
VREAKKQVEMAL

SEQ ID NO: 13 - protein, 12 aa, Artificial Sequence
  synthetic peptide
SNEAKNQVEKAL

SEQ ID NO: 14 - protein, 12 aa, Artificial Sequence
  synthetic peptide
SNEAKKQVERAL

SEQ ID NO: 15 - protein, 12 aa, Artificial Sequence
  synthetic peptide
SNEAKKQVEMAL

SEQ ID NO: 16 - protein, 12 aa, Artificial Sequence
  synthetic peptide
SDEAKNQVEKAL

SEQ ID NO: 17 - protein, 12 aa, Artificial Sequence
  synthetic peptide
SDEAKKQVERAL

SEQ ID NO: 18 - protein, 12 aa, Artificial Sequence
  synthetic peptide
SDEAKKQVEMAL

SEQ ID NO: 19 - protein, 12 aa, Artificial Sequence
  synthetic peptide
SREAKNQVERAL

SEQ ID NO: 20 - protein, 12 aa, Artificial Sequence
  synthetic peptide
SREAKNQVEMAL

SEQ ID NO: 21 - protein, 7 aa, Artificial Sequence
  synthetic peptide
MKQLEDK

SEQ ID NO: 22 - protein, 7 aa, Artificial Sequence
  synthetic peptide
VKQLEDK

SEQ ID NO: 23 - protein, 28 aa, Artificial Sequence
  synthetic peptide
QLEDKVKQLRRDLDASREAKNELQDKVK

SEQ ID NO: 24 - protein, 28 aa, Artificial Sequence
  synthetic peptide
LEDKVKQARRDLDASREAKNELQDKVKQ

SEQ ID NO: 25 - protein, 28 aa, Artificial Sequence
  synthetic peptide
EDKVKQAERDLDASREAKNQLQDKVKQL

SEQ ID NO: 26 - protein, 28 aa, Artificial Sequence
  synthetic peptide
DKVQKAEDDLDASREAKNQVQDKVKQLE

SEQ ID NO: 27 - protein, 28 aa, Artificial Sequence
  synthetic peptide
KVKQAEDKLDASREAKNQVEDKVKQLED

SEQ ID NO: 28 - protein, 29 aa, Artificial Sequence
  synthetic peptide
VKQAEDKVDASREAKNQVEXKKVKQLEDK

SEQ ID NO: 29 - protein, 29 aa, Artificial Sequence
  synthetic peptide
KQAEDKVKASREAKNQVEXKAVKQLEDKV

SEQ ID NO: 30 - protein, 28 aa, Artificial Sequence
  synthetic peptide
QAEDKVKQSREAKNQVEXALKQLEDKVQ

SEQ ID NO: 31 - protein, 29 aa, Artificial Sequence
  synthetic peptide
KQAEDKVKASREAKNQVEXALEQLEDKVK

SEQ ID NO: 32 - protein, 21 aa, Artificial Sequence
  synthetic peptide
LRRDLDAENEAKKQVEKALEC

SEQ ID NO: 33 - protein, 21 aa, Artificial Sequence
  synthetic peptide
LRRDLDAEDEAKKQVEKALEC

SEQ ID NO: 34 - protein, 21 aa, Artificial Sequence
  synthetic peptide
LRRDLDAEREAKNQVEKALEC

SEQ ID NO: 35 - protein, 21 aa, Artificial Sequence
  synthetic peptide
LRRDLDAEREAKKQVERALEC

SEQ ID NO: 36 - protein, 21 aa, Artificial Sequence
  synthetic peptide
LRRDLDAEREAKKQVEMALEC

SEQ ID NO: 37 - protein, 21 aa, Artificial Sequence
  synthetic peptide
LRRDLDAVNEAKKQVEKALEC

SEQ ID NO: 38 - protein, 21 aa, Artificial Sequence
  synthetic peptide
LRRDLDAVDEAKKQVEKALEC

SEQ ID NO: 39 - protein, 21 aa, Artificial Sequence
  synthetic peptide
LRRDLDAVREAKNQVEKALEC

SEQ ID NO: 40 - protein, 21 aa, Artificial Sequence
  synthetic peptide
LRRDLDAVREAKKQVERALEC

SEQ ID NO: 41 - protein, 21 aa, Artificial Sequence
  synthetic peptide
LRRDLDAVREAKKQVEMALEC

SEQ ID NO: 42 - protein, 21 aa, Artificial Sequence
  synthetic peptide
LRRDLDASNEAKNQVEKALEC

SEQ ID NO: 43 - protein, 21 aa, Artificial Sequence
  synthetic peptide
LRRDLDASNEAKKQVERALEC

SEQ ID NO: 44 - protein, 21 aa, Artificial Sequence
  synthetic peptide
LRRDLDASNEAKKQVEMALEC

SEQ ID NO: 45 - protein, 21 aa, Artificial Sequence
  synthetic peptide
LRRDLDASDEAKNQVEKALEC

SEQ ID NO: 46 - protein, 21 aa, Artificial Sequence
  synthetic peptide
LRRDLDASDEAKKQVERALEC

SEQ ID NO: 47 - protein, 21 aa, Artificial Sequence
  synthetic peptide
LRRDLDASDEAKKQVEMALEC

SEQ ID NO: 48 - protein, 21 aa, Artificial Sequence
  synthetic peptide
LRRDLDASREAKNQVERALEC

SEQ ID NO: 49 - protein, 21 aa, Artificial Sequence
  synthetic peptide
LRRDLDASREAKNQVEMALEC

(19) United States
(12) Patent Application Publication
(10) Pub. No.: US 2009/0162369 A1
(43) Pub. Date: Jun. 25, 2009

(54) SYNTHETIC CHIMERIC PEPTIDES

(76) Inventors: Katin Helena Threse Nordstrom, Genarp (SE); Michael F. Good, The Gap (AU); Michael R. Batzloff, Durack (AU)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/333,222

(22) Filed: Dec. 11, 2008

Related U.S. Application Data
(60) Provisional application No. 61/007,570, filed on Dec. 12, 2007.

(51) Int. Cl. A61K 39/395; C07K 14/00; C07K 5/00; A61K 39/09; C07K 16/18; G01N 33/569

(52) U.S. Cl. 424/139.1; 530/326; 530/331; 530/330; 530/329; 530/328; 530/327; 424/190.1; 530/387.9; 530/387.3; 435/7.34

(57) ABSTRACT

The present invention relates generally to chimeric peptides comprising one or more protective epitopes in a conformation enabling immunological interactivity and to vaccine compositions comprising same. The present invention is particularly directed to a chimeric peptide capable of inducing protecting antibodies against Group A streptococci.

### CROSS-REFERENCE TO RELATED APPLICATION

This application claims priority benefit of U.S. provisional application Ser. No. 61/007,570, filed Dec. 12, 2007, which application is incorporated herein by reference in it entirety.

### TECHNICAL FIELD OF THE INVENTION

The present invention relates generally to chimeric peptides comprising one or more protective epitopes in a conformation enabling immunological interactivity and to vaccine compositions comprising the same. The present invention is particularly directed to a chimeric peptide capable of inducing protective antibodies against Group A streptococci (GAS).

### BACKGROUND TO THE INVENTION

A List of References of the publications referred to in this specification by author are collected at the end of the description. Sequence Identity Numbers (SEQ ID NOs.) for the amino acid sequences referred to in the specification are defined following the bibliography.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

The coiled coil structure is an important structural and biologically abundant motif found in a diverse group of proteins (Cohen and Parry, 1990, 1986) and many proteins which may be useful vaccine candidates against various diseases have been found to possess a coiled coil structure. More than 200 proteins have now been predicted to contain coiled coil domains (Lupas et al., 1991). These include surface proteins of certain bacteria such as streptococcal protein A and M proteins; viruses such as influenza hemagglutinin and human immunodeficiency virus (HIV) glycoprotein gp45; and protozoa such as VSG of Trypanosomes. All coiled coil motifs share a characteristic seven amino acid residue repeat (a-b-c-d-e-f-g)ₙ. The X-ray structure of several coiled coil domains have been solved and these include the leucine zipper portion of the yeast transcription factor GCN4 dimer (O'Shea et al. 1991), the repeat motif of 1-spectrin (Yan, 1993), together with the GCN4 leucine zipper trimer (Harbury et al., 1994) and tetramer (Harbury et al., 1993) mutants.

In the development of a subunit vaccine based on these proteins, it is generally difficult to map epitopes within the coiled coil structure. Furthermore, protective epitopes may need to be presented in the correct conformation for immunological recognition, such as antibody binding. This is especially important in defining a stable minimal epitope and using it as a vaccine.

Streptococcus pyogenes, also known as Group A Streptococcus (GAS), is a serious human pathogen capable of causing a variety of human diseases ranging from uncomplicated pharyngitis and pyoderma to severe life threatening invasive infections. Infections caused by GAS range from uncomplicated skin and soft tissue infections to life threatening invasive diseases such as bacteremia and necrotizing fasciitis as well as non-supperative sequalae such as rheumatic fever, rheumatic heart disease and acute glomerulonephritis.

Infections due to GAS represent a public health problem of major proportions in both developing and developed countries. Illness attributable to GAS infection results in a huge burden to health care systems worldwide, as there are an estimated over 25-35 million infections per year in the US alone. Although uncomplicated pharyngitis and skin and soft-tissue infections account for most of these infections, there is a resurgence in the incidence of the life-threatening illnesses, such as necrotizing fasciitis and toxic shock syndrome, in hospitals and other institutions. Uncomplicated infection can also lead to serious sequelae, such as, acute rheumatic fever (ARF) and glomerulonephritis. Acute rheumatic fever continues to be a leading cause of heart disease worldwide. WHO estimates that GAS causes 517,000 deaths worldwide annually, with approximately one-third of those deaths (163,000) related to invasive GAS disease and the remainder (354,000) related to nonsuppurative sequelae of GAS infections (Bisno et al., 2005).

Presently, penicillin is still used as a first-line therapy in the treatment of most GAS infections. Currently available methods of prevention are either inadequate or ineffective, as evidenced by the morbidity and mortality still associated with this pathogen worldwide. The current situation with respect to the health care burden and the treatment and prevention of GAS infections therefore warrants the development of other preventative/therapeutic treatment measures.

The surface M protein is the major virulence determinant and protective antigen of GAS. In the immune host, M protein antibodies are opsonic and promote ingestion and killing of GAS by phagocytic cells. The M proteins of GAS isolates are multivalent and may elicit the production of antibodies that cross-react with human tissues.

M protein contain a seven-residue periodicity which strongly suggested that the central rod region of the molecule is in a coiled coil conformation (Manula and Fischetti, 1980). Overlapping peptides were made that spanned this region (see WO 93/21220) and mouse antibodies raised against one synthetic 20mer peptide (designated “p145”) from the highly conserved C-terminal region can opsonise and kill multiple isolates of GAS (Pruksakorn et al., 1994a). In addition, p145 can inhibit in vitro killing mediated by human sera. Of concern is that p145 may also stimulate heart cross-reactive T cells (Pruksakorn et al., 1992; 1994b). The B cell epitope within p145 was thought to be conformational because truncated peptides fail to elicit a protective antibody response (Pruksakorn, 1994).

Non-host reactive, conformationally constrained, minimal B cell epitopes of the GAS M protein have now been identified (WO 96/11944; Heyman et al. 1997). WO 96/11944 describes chimeric peptides in which a first amino acid sequence comprising a conformational epitope is embedded within a second amino acid sequence, wherein the first and second amino acid sequences are derived from peptides, polypeptides or proteins having similar native conformations. The second amino acid sequence provides a “framework” for the first amino acid sequence. WO96/11944 discloses mapping the conformational epitope within the p145 peptide from the conserved C-terminal region of the GAS M protein (e.g. Example 14). Chimeric peptides were constructed in which a 12 amino acid window of p145 sequence was inserted into the leucine zipper motif in GCN4, the DNA binding protein of yeast (O'Shea et al., 1991). The 12 amino acid window of peptide 145 sequence was inserted into the so-called “Jcon” peptide derived from leucine zipper motif in GCN4 in such a way as to preserve any potential helical structure. The window was shifted one residue at a time to give nine peptides (referred to as J1→J9) that represented the entire p145 sequence.

There is still a need for improved vaccines, for example, against GAS associated diseases. In addition, poor vaccination uptake, the on-going health risk for non-immunized individuals infected with GAS, and the possible ill-health in immunized individuals in the early phase of a sever acute GAS infection e.g., before an immune response is mounted, make other therapeutic and prophylactic approaches for the treatment of GAS infection desirable. Such therapies may be adjunct to other treatments such as vaccination or antibiotics, or stand-alone therapies.

### SUMMARY OF THE INVENTION

The present inventors have now found that chimeric structures based on the p145 amino acid sequence inserted with a framework structure comprising a second amino acid sequence provide unexpectedly improved immunogenicity. Furthermore, the chimeric peptides have also been found to be protective against multiple strains of the same streptococcal Grouping, e.g. Group A streptococci. In addition, it is contemplated that the chimeric peptides of the present invention will be useful against multiple Groups of streptococci, i.e. in addition to the Group A, also, for example, Group C and/or Group G.

Accordingly, the present invention provides a chimeric peptide comprising a first amino acid sequence comprising a conformational epitope inserted within a second amino acid sequence wherein the first and second amino acid sequences are derived from peptides, polypeptides or proteins having similar native conformations, wherein the first amino acid sequence has at least three amino acids selected from within the following sequence:

(SEQ ID NO: 1)

L-R-R-D-L-D-A-X¹-X²-E-A-K-X³-Q-V-E-X⁴-A-L-E

wherein
X¹ is selected from S, E and V;
X² is selected from R, N and D;
X³ is selected from K and N; and
X⁴ is selected from K, R and M,
   wherein the at least three amino acids constitute a conformational epitope and wherein at least one of the at least three amino acids is selected from the group consisting of X¹ being E or V, X² being N or D, X³ being N and X⁴ being R or M.

The present invention further provides a vaccine useful against streptococci, the vaccine comprising a chimeric peptide according to the first aspect and one or more pharmaceutically acceptable carriers and/or excipients.

The vaccine may further comprise an adjuvant and/or other immune stimulating molecules

The present invention further provides a vaccine composition useful in the development of humoral immunity to a streptococcal M protein but minimally cross-reactive with heart tissue, the vaccine comprising a chimeric peptide according to the first aspect and one or more pharmaceutically acceptable carriers and/or excipients.

The present invention also provides an antibody which binds immunospecifically to a conformational epitope (e.g. B-cell epitope) of a streptococcal, preferably a GAS, M-protein, the conformational epitope comprising at least three amino acids from within SEQ ID NO:1 wherein X¹ is selected from S, E and V; X² is selected from R, N and D; X³ is selected from K and N; and X⁴ is selected from K, R and M, and wherein at least one amino acid of the at least three amino acids is selected from the group consisting of X¹ being E or V, X² being N or D, X³ being N and X⁴ being R or M.

The term antibody also encompasses antibody fragments and antibody conjugates as described herein.

The present invention also provides a pharmaceutical composition comprising one or more antibodies, antibody fragments or antibody conjugates according to the present invention and one or more pharmaceutically acceptable carriers and/or excipients.

The present invention also provides a method of treating or ameliorating streptococcal, and in particular GAS, infection in a human or other mammalian subject comprising administering to the subject a composition according to the invention, wherein the composition is administered in an amount effective to prevent an increase in bacterial count or to reduce bacterial count in a sample from the subject.

The present invention also provides a method of neutralizing a streptococcal, and in particular GAS, pathogen in a subject exposed to the pathogen comprising administering to the subject infected with the streptococcal, and in particular GAS, pathogen a pharmaceutical composition according to the invention, wherein the composition is administered in an amount effective to opsonize the pathogen in the serum of the subject.

The present invention also provides a method of maintaining a therapeutically or prophylactically effective serum titre of an antibody against a streptococcal M protein, and in particular an M-protein of GAS, in a subject, the method comprising administering a plurality of doses of a pharmaceutical composition according to the invention, wherein each of the doses is administered in an amount effective to prevent an increase in bacterial count or to reduce bacterial count in a sample from the subject and/or to reduce the severity of one or more disease symptoms or to prevent onset of one or more diseases arising from a streptococcal, and in particular a GAS, infection, and/or to opsonize the pathogen in the serum of the subject.

The present invention also provides a method of treating or ameliorating a streptococcal, and in particular GAS, infection in a human or other mammalian subject or preventing, ameliorating or treating a disease or complication associated with streptococcal, and in particular GAS, infection of a human or other mammalian subject or neutralizing a streptococcal, and in particular a GAS, pathogen in a subject exposed to the pathogen, the method comprising administering or recommending administration of composition according to the invention to a subject previously identified as suffering from a streptococcal, and in particular a GAS, infection or a disease or complication associated with streptococcal, and in particular GAS, infection or at risk of developing a streptococcal, and in particular a GAS, infection or a disease or complication associated with streptococcal, and in particular GAS, infection.

The present invention also provides an antibody according to the present invention, or nucleic acid encoding the antibody, or a cell expressing the antibody, or a composition comprising the antibody, nucleic acid, or cell, for use in medicine.

The present invention also provides a chimeric peptide according to the present invention for use in medicine.

The present invention also provides for the use of an antibody according to the present invention, or nucleic acid encoding the antibody, or a cell expressing the antibody, in the manufacture of a medicament for the treatment of a streptococcal infection or a disease or condition associated with a streptococcal infection, and in particular a GAS infection.

Compositions, including medicaments, of the present invention are useful, for example, in the treatment of an acute severe streptococcal, and in particular GAS, infection or a disease or complication associated therewith, such as uncomplicated pharyngitis, pyoderma, skin infection, soft tissue infection, bacteremia, necrotizing fasciitis, rheumatic fever, rheumatic heart disease, acute glomerulonephritis, or morbidity.

The present invention also provides a method for the diagnosis of streptococcal infection in a subject comprising contacting a biological sample from the subject with an antibody binding effective amount of a chimeric peptide for a time and under conditions sufficient for an antibody-chimeric peptide complex to form, and then detecting the complex.

### BRIEF DESCRIPTION OF THE FIGURES

FIG. 1: Reactivity of anti-p145 mouse antisera against Peptide Nos. 1-19. Reactivity is plotted as a mean absorbance value at 405 nm. Data for p145 and peptides 2-10 are indicated by solid lines; data for peptides 11-18 are indicated by dashed lines.

FIG. 2: IgG titres in murine antisera raised to Peptide Nos: 1-19.

FIG. 3: Binding of purified anti-peptide (Nos 1-19) antibodies to the p145 peptide. Top panel: data for p145, peptides 2-3, 7, and 11 are indicated by solid lines; data for peptides 5 and 9-10 are indicated by dashed lines. Bottom panel: data for p145, peptides 12, 13, 14, and 16 are indicated by solid lines; data for peptides 18-19 are indicated by dashed lines.

FIG. 4: Binding of purified anti-peptide (Nos: 2, 3, 5, 7-19) antibodies to GAS

FIG. 5: Binding of purified anti peptide No: 18 antibody to GAS strains 2031, 1036 and 88/30.

FIG. 6: Dose-dependent curves of the binding of anti-peptide No. 18 antibody to GAS 2031.

FIGS. 7A-7C: Binding of anti-peptide Nos: 1-19 antisera to a series of p145-derived peptides.

FIG. 8: Peptide specific titres in murine sera samples on day 35, after primary immunization with TN18, TN19, J18 and J19 peptides conjugated to diphtheria toxoid (DT) and two boosters.

FIG. 9: The geometric mean of peptide-specific serum IgG titres of B10.BR mice immunised with different peptides conjugated to diphtheria toxoid (DT). Control mice were immunised with vehicle (PBS). Five mice were immunised per group and error bars show the standard error of the mean (SEM). The highest titres were obtained after primary immunisation plus two booster injections. Although titres had decreased 43 days after the final boost (Day 71), they remained reasonably high.

FIG. 10: The geometric mean of DT-specific serum IgG titres of b10.BR mice immunised with different peptides conjugated to DT. Control mice were immunised with vehicle (PBS). Five mice were immunised per group and error bars show the standard error of the mean (SEM).

FIG. 11: Binding of anti-peptide antisera from mice immunized with different peptides conjugated to DT to the p145 peptide. Bold underlined letters indicate amino acid positions that were changed and found to enhance immunogenicity. Underlined letters indicate the second amino acid framework peptide sequence.

FIG. 12: Binding of anti-peptide antisera from mice immunized with different peptides conjugated to DT to the J8 peptide. Bold underlined letters indicate amino acid positions that were changed and found to enhance immunogenicity. Underlined letters indicate the second amino acid framework peptide sequence.

FIG. 13: In vitro opsonisation of 88/30 GAS by immune sera. Chart shows percent opsonisation of GAS by pooled sera compared to normal mouse sera.

### DETAILED DESCRIPTION OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The following single and three letter abbreviations are used for amino acid residues:

| Amino Acid | Three-letter | |
|---|---|---|
| Abbreviation | Symbol | One-letter |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any residue | Xaa | X |

### Chimeric Peptides

The present invention provides a chimeric peptide comprising a first amino acid sequence comprising a conformational epitope inserted within a second amino acid sequence wherein the first and second amino acid sequences are derived from peptides, polypeptides or proteins having similar native conformations, wherein the first amino acid sequence has at least three amino acids selected from within the following sequence:

(SEQ ID NO: 1)

L-R-R-D-L-D-A-X¹-X²-E-A-K-X³-Q-V-E-X⁴-A-L-E

wherein
X¹ is selected from S, E and V;
X² is selected from R, N and D;
X³ is selected from K and N; and
X⁴ is selected from K, R and M,
   wherein the at least three amino acids constitute a conformational epitope and wherein at least one amino acid of the at least three amino acids is selected from the group consisting of X¹ being E or V, X² being N or D, X³ being N and X⁴ being R or M.

The second amino acid sequence constitutes a “framework peptide” and provides an appropriate conformation for the chimeric peptide. The conformational epitope which is present in the first amino acid sequence in its native (natural) state is not present in the first amino acid sequence when in an isolated (non-native) state. The framework peptide is selected or otherwise engineered to impart a similar conformation upon the first amino acid sequence as present in its naturally occurring form, e.g. in its protein form.

First amino acid sequence: It is particularly preferred that the conformational epitope in the first amino acid sequence be presented in a coiled coil conformation. Therefore, it is particularly preferred that the framework amino acid sequence assume a coiled coil conformation so as to make it useful in presenting epitopes present in the first amino acid sequence in a similar conformation, i.e. a coiled coil confirmation.

Preferred coil coiled structures are α-helical coiled coil structures. As such, in a preferred embodiment of the first aspect, the second amino acid sequence folds to an α-helical coiled coil conformation.

The conformational epitope is preferably a B-cell epitope. The conformational epitope comprises at least 3, more preferably at least 4, yet more preferably at least 5 and most preferably at least 6 amino acid residues from within the first amino acid sequence.

In a preferred embodiment, the first amino acid sequence has at least three amino acids selected from within the following sequence: X¹-X²-E-A-K-X³-Q-V-E-X⁴-A-L (SEQ ID NO:2). Chimeric peptides based on SEQ ID NO:2 have been found to provide particularly improved immunogenicity and cross-strain immunological interactivity.

At least one amino acid of the at least three amino acids constituting the conformational epitope is selected from the group consisting of X¹ being E or V, X² being N or D, X³ being N and X⁴ being R or M. In a preferred embodiment, the at least one amino acid is selected from the group consisting of X³ being N and X⁴ being R or M. More preferably, the conformational epitope comprises at least X³ being N and X⁴ being R or M. Yet more preferably, the conformational epitope comprises X¹ being S, X² being R, X³ being N and X⁴ being R or M.

Preferably, the first amino acid sequence comprises at least 5, more preferably at least 6, yet more preferably at least 7, yet more preferably at least 8, yet more preferably at least 9 and yet more preferably at least 10 contiguous amino acid residues of SEQ ID NO:1 or SEQ ID NO:2. The first amino acid sequence may suitably comprise at least 15 contiguous amino acid residues of SEQ ID NO:1.

Alternatively, non-contiguous amino acids may be selected such as those on the outside face of the helix and which are required or sufficient for activity.

In a preferred embodiment, the first amino acid sequence comprises from 10 to 15 contiguous amino acid residues, preferably 12 contiguous amino acid residues, of SEQ ID NO:1. In a particularly preferred embodiment, the first amino acid sequence comprises the 12 contiguous amino acid residues with the following sequence: X¹-X²-E-A-K-X²-Q-V-E-X⁴-A-L (SEQ ID NO:2). Chimeric peptides based on SEQ ID NO:2 have been found to provide particularly improved immunogenicity and cross-strain immunological interactivity.

Examples of suitable first amino acid sequences comprising 12 contiguous amino acid residues according to SEQ ID NO:2 include:

| | |
|---|---|
| ENEAKKQVEKAL | (SEQ ID NO: 3) |
| EDEAKKQVEKAL | (SEQ ID NO: 4) |
| EREAKNQVEKAL | (SEQ ID NO: 5) |
| EREAKKQVERAL | (SEQ ID NO: 6) |
| EREAKKQVEMAL | (SEQ ID NO: 7) |
| VNEAKKQVEKAL | (SEQ ID NO: 8) |
| VDEAKKQVEKAL | (SEQ ID NO: 9) |
| VREAKNQVEKAL | (SEQ ID NO: 10) |
| VREAKKQVERAL | (SEQ ID NO: 11) |
| VREAKKQVEMAL | (SEQ ID NO: 12) |
| SNEAKNQVEKAL | (SEQ ID NO: 13) |
| SNEAKKQVERAL | (SEQ ID NO: 14) |
| SNEAKKQVEMAL | (SEQ ID NO: 15) |
| SDEAKNQVEKAL | (SEQ ID NO: 16) |
| SDEAKKQVERAL | (SEQ ID NO: 17) |
| SDEAKKQVEMAL | (SEQ ID NO: 18) |
| SREAKNQVERAL | (SEQ ID NO: 19) |
| SREAKNQVEMAL | (SEQ ID NO: 20) |

Preferred amino acid sequences are SEQ ID NOs: 19 and 20.

Second amino acid sequence: If an epitope is known to reside within a particular protein structural conformation, such as a I-helical coiled coil, then a model peptide can be synthesised to fold to this conformation. This peptide will become the framework peptide.

It is preferred that the second amino acid sequence which has a similar conformation to the first amino acid sequence in its native state be derived from a completely unrelated protein, polypeptide or peptide.

Alternatively, although less preferred, the second amino acid sequence which has a similar conformation to the first amino acid sequence in its native state is derived from a related protein, polypeptide or peptide. For example, in the present case, where the first amino acid sequence is based on amino acids within the p145 amino acid sequence being inserted with a “framework” second amino acid sequence, the second amino acid sequence may be derived from the other streptococcal proteins. In particular, the second amino acid sequence may be derived from a streptococcal M-protein, more particularly from the M-protein of GAS. The second amino acid sequence may comprise one or more amino acids from the N and/or C-terminals of p145. For example, the 7 amino acid N-terminal fragment of p145 (SEQ ID NO:1) may constitute one part of the framework amino acid sequence (e.g. the N-terminal “flanking region” of the chimeric peptide).

Model peptides that fold into a coiled coil, for example an I-helical coiled coil, have been studied have been well studied. In the design of a parallel two-stranded coiled coil motif (a-b-c-d-e-f-g)ₙ, several general considerations are important (Cohen and Parry, 1990).

The construction of a framework peptide is suitably based on the seven amino acid residue repeat:
(a-b-c-d-e-f-g)ₙ
   where a and d positions preferably have large a polar residues, positions b, c and f are generally polar and charged and positions e and g generally favour interchain ionic interactions (e.g. the acid/base pair of Glu/Lys). It is also known that when positions a and d are occupied by V and L, or I and L, a coiled coil dimer is favoured whereas I and I favours trimer formation and L and I favour tetramer interactions (Harbury et al. 1994).

A particularly preferred framework peptide can be designed and based on the structure of a peptide corresponding to GCN4 leucine zipper (O'Shea et al., 1989; 1991) or its trimer (Harbury et al., 1994) or tetramer (Harbury et al., 1993) and the repeat motif of Ispectrin (Yan, 1993). The GCN4 leucine zipper is particularly preferred.

A model I-helical coiled coil peptide based on the structure of a peptide corresponding to the GCN4 leucine zipper (O'Shea et al 1989, 1991) has a seven residue leucine repeat (in the d position) and a consensus valine (in the α position). The first heptad contains the sequence: M K Q L E D K [SEQ ID NO:21] which includes several of the features found in a stable coiled coil heptad repeat. These include an acid/base pair (glu/lys) at positions e and g, and polar groups in positions b, c, f.

Another model heptad repeat is derived from the consensus features of the GCN4 leucine zipper peptide: V K Q L E D K [SEQ ID NO:22], which when repeated would give a model peptide, (V K Q L E D K)ₙ, with the potential to form a I-helical coiled coil.

Where required, the framework peptide may be longer than the four repeats.

Overlapping fragments of the first amino acid sequence comprising the conformational epitope are embedded within the framework second amino acid sequence. For example, a coiled coil (e.g. α-helical coiled coil) conformational epitope would be embedded between flanking peptides derived from a completely unrelated protein with a similar native conformation. The resulting chimeric peptides can be tested for immunological activity, i.e. antigenicity (recognition by mAb) or immunogenicity (production of appropriate antibody response).

Examples of chimeric peptide sequences in which the second amino acid is derived from a completely unrelated protein include:

| | |
|---|---|
| QLEDKVKQLRRDLDASREAKNELQDKVK; | (SEQ ID NO: 23) |
| LEDKVKQARRDLDASREAKNELQDKVKQ; | (SEQ ID NO: 24) |
| EDKVKQAERDLDASREAKNQLQDKVKQL; | (SEQ ID NO: 25) |
| DKVQKAEDDLDASREAKNQVQDKVKQLE; | (SEQ ID NO: 26) |
| KVKQAEDKLDASREAKNQVEDKVKQLED; | (SEQ ID NO: 27) |
| VKQAEDKVDASREAKNQVEX⁵KKVKQLEDK; | (SEQ ID NO: 28) |
| KQAEDKVKASREAKNQVEX⁵KAVKQLEDKV; | (SEQ ID NO: 29) |
| QAEDKVKQSREAKNQVEX⁵ALKQLEDKVQ; | (SEQ ID NO: 30) |
| and | |
| KQAEDKVKASREAKNQVEX⁵ALEQLEDKVK | (SEQ ID NO: 31) |

wherein X⁵ is selected from K, R and M

Examples of chimeric peptide sequences in which the second amino acid is derived from a related protein include:

| | |
|---|---|
| LRRDLDAENEAKKQVEKALEC; | (SEQ ID NO: 32) |
| LRRDLDAEDEAKKQVEKALEC; | (SEQ ID NO: 33) |
| LRRDLDAEREAKNQVEKALEC; | (SEQ ID NO: 34) |
| LRRDLDAEREAKKQVERALEC; | (SEQ ID NO: 35) |
| LRRDLDAEREAKKQVEMALEC; | (SEQ ID NO: 36) |
| LRRDLDAVNEAKKQVEKALEC; | (SEQ ID NO: 37) |
| LRRDLDAVDEAKKQVEKALEC; | (SEQ ID NO: 38) |
| LRRDLDAVREAKNQVEKALEC; | (SEQ ID NO: 39) |
| LRRDLDAVREAKKQVERALEC; | (SEQ ID NO: 40) |
| LRRDLDAVREAKKQVEMALEC; | (SEQ ID NO: 41) |
| LRRDLDASNEAKNQVEKALEC; | (SEQ ID NO: 42) |
| LRRDLDASNEAKKQVERALEC; | (SEQ ID NO: 43) |
| LRRDLDASNEAKKQVEMALEC; | (SEQ ID NO: 44) |
| LRRDLDASDEAKNQVEKALEC; | (SEQ ID NO: 45) |
| LRRDLDASDEAKKQVERALEC; | (SEQ ID NO: 46) |
| LRRDLDASDEAKKQVEMALEC; | (SEQ ID NO: 47) |
| LRRDLDASREAKNQVERALEC; | (SEQ ID NO: 48) |
| and | |
| LRRDLDASREAKNQVEMALEC; | (SEQ ID NO: 49) |

Analogues: It will be understood that reference herein to the chimeric peptides of the present invention also includes analogues thereof unless specifically stated otherwise. The term “analogues” extends to any functional, chemical or recombinant equivalent of the peptides of the present invention characterised, in a most preferred embodiment, by their possession of at least one B cell epitope from the M protein of GAS, wherein an antibody reactive to the B cell epitope is only minimally reactive with human heart tissue. The term “analogue” is also used herein to extend to any amino acid derivative of the peptides described above.

Analogues of the peptides described herein include, but are not limited to, modifications to side chains, incorporation of unnatural amino acids and/or their derivatives during peptide synthesis and the use of crosslinkers and other methods which impose conformational constraints on the polypeptides or their analogues.

The chimeric peptides of the present invention may by chemically modified. Chemical modification may be useful for improving the in vivo efficacy of the peptides since the unmodified peptides may not have a sufficiently long serum and/or tissue half-life. Chemical modification of the subject peptides may also be important to improve their antigenicity including the ability for certain regions of the peptides to act as B and/or T cell epitopes.

Examples of side-chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5′-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids.

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{β}-methylamino acids, introduction of double bonds between C_{α} and C_{β} atoms of amino acids and the formation of cyclic peptides or analogues by introducing covalent bonds such as forming an amide bond between the N and C termini, between two side chains or between a side chain and the N or C terminus.

Analogues also encompasses peptides of the present invention in which amino acids have undergone “conservative substitution”. Conservative substitution refers to a change in the amino acid composition of the polypeptide that does not substantially alter the polypeptides activity. Thus, “conservatively modified variations” of a particular amino acid sequence refers to amino acid substitutions of those amino acids that are not critical for peptide activity or substitution of amino acids with other amino acids having similar properties (e.g., acidic, basic, positively or negatively charged, polar or non-polar) such that the substitutions of even critical amino acids do not substantially alter activity. Conservative substitution tables providing functionally similar amino acids are well-known in the art (Creighton, 1984). In addition, individual substitutions, deletions or additions which alter, add or delete a single amino acid or a small percentage of amino acids in an encoded sequence are also “conservatively modified variations.”

Preferably, the chimeric peptides of the present invention comprise SEQ ID NO:2. More preferably, the first amino acid sequence essentially consists of SEQ ID NO: 19 or 20.

### Production of Chimeric Peptides

The chimeric peptides of the present invention may be produced by recombinant means or may be chemically synthesised by, for example, the stepwise addition of one or more amino acid residues in defined order using solid phase peptide synthesis (SPPS) techniques. The peptides may comprise naturally occurring amino acid residues or may also contain non-naturally occurring amino acid residues such as certain D-isomers or chemically modified naturally occurring residues. These latter residues may be required, for example, to facilitate or provide conformational constraints and/or limitations to the peptides. The selection of a method of producing the chimeric peptides will depend on factors such as the required type, quantity and purity of the peptides as well as ease of production and convenience.

The chimeric peptides are preferably relatively short. This lends the peptides to be readily synthesizable using well-known SPPS techniques (Merrifield, 1963).

Alternatively, the chimeric peptides can be prepared using well known recombinant techniques in which a nucleotide sequence encoding the polypeptide of interest is expressed in cultured cells such as described in Ausubel et al. (1987) and in Sambrook et al. (1989), both of which are incorporated herein by reference in their entirety.

Typically, nucleic acids encoding the desired chimeric peptides are used in expression vectors. The phrase “expression vector” generally refers to nucleotide sequences that are capable of affecting expression of a gene in hosts compatible with such sequences.

These expression vectors typically include at least suitable promoter sequences and optionally, transcription termination signals. Additional factors necessary or helpful in effecting expression may also be used as described herein.

Nucleic acid encoding the chimeric peptides of the present invention will typically be incorporated into DNA constructs capable of introduction into and expression in an in vitro cell culture. Specifically, DNA constructs will be suitable for replication in a prokaryotic host, such as bacteria, e.g. E. coli, or may be introduced into a cultured mammalian, plant, insect, yeast, fungi or other eukaryotic cell lines.

DNA constructs prepared for introduction into a particular host will typically include a replication system recognized by the host, the intended DNA segment encoding the desired polypeptide, and transcriptional and translational initiation and termination regulatory sequences operably linked to the polypeptide encoding segment. A DNA segment is “operably linked” when it is placed into a functional relationship with another DNA segment.

For example, a promoter or enhancer is operably linked to a coding sequence if it stimulates the transcription of the sequence. DNA for a signal sequence is operably linked to DNA encoding a polypeptide if it is expressed as a preprotein that participates in the secretion of the peptide. Generally, DNA sequences that are operably linked are contiguous, and, in the case of a signal sequence, both contiguous and in reading phase. However, enhancers need not be contiguous with the coding sequences whose transcription they control. Linking is accomplished by ligation at convenient restriction sites or at adapters or linkers inserted in lieu thereof.

The selection of an appropriate promoter sequence generally depends upon the host cell selected for the expression of the DNA segment. Examples of suitable promoter sequences include prokaryotic, and eukaryotic promoters well-known in the art. The transcriptional regulatory sequences will typically include a heterologous enhancer or promoter which is recognized by the host. The selection of an appropriate promoter will depend upon the host, but promoters such as the trp, lac and phage promoters, tRNA promoters and glycolytic enzyme promoters are known and available.

Conveniently available expression vectors which include the replication system and transcriptional and translational regulatory sequences together with the insertion site for the peptide encoding segment may be employed. Examples of workable combinations of cell lines and expression vectors are described in Sambrook et al. (1989) and in Metzger et al. (1988). For example, suitable expression vectors may be expressed in, e.g., insect cells, e.g., Sf9 cells, mammalian cells, e.g., CHO cells and bacterial cells, e.g., E. coli.

In certain instances, the chimeric peptides of the invention are produced by expression in transgenic animals (i.e., non-human animals containing an exogenous DNA sequence in the genome of germ-line and somatic cells introduced by way of human intervention) such as bovines, goats, rabbits, sheep, pigs or mice. Methods for production of recombinant polypeptides by transgenic non-human species are known in the art and are described, for example, in U.S. Pat. Nos. 5,304,489, 5,633,076 and 5,565,362 which are incorporated herein by reference in their entirety, as well as in PCT publications PCT/US93/05724 and PCT/US95/09580, both of which are incorporated herein by reference in their entirety. An advantage of the transgenic animals is the isolation of the polypeptides of interest in large amounts, especially by economical purification methods. For example, the production of transgenic bovine species containing a transgene encoding a human lactoferrin polypeptide targeted for expression in mammary secreting cells is described in WO 91/08216, incorporated herein by reference in its entirety. When lactoferrin variants are produced in transgenic bovines the human protein typically is separated from the bovine milk proteins (e.g., whey proteins, caseins, bovine lactoferrin, IgA, albumin. lysozyme, ss-lactoglobulin) before use (e.g., administration to humans). Alternatively, use may be made of whole or partially purified bovine milk containing the desired peptide.

Another method for preparing the chimeric peptides of the invention is to employ an in vitro transcription/translation system. DNA encoding a peptide of the invention is cloned into an expression vector as described above. The expression vector is then transcribed and translated in vitro. The translation product can be used directly or first purified. Peptides resulting from in vitro translation typically do not contain the post-translation modifications present on polypeptides synthesized in vivo. Methods for synthesis of peptides by in vitro translation are described by, for example, Berger & Kimmel (1987).

The term “isolated”, “purified” or “substantially pure” means an object species is the predominant macromolecular species present (i.e., on a molar basis it is more abundant than any other individual species in the composition), and preferably the object species comprises at least about 50 percent (on a molar basis) of all macromolecular species present. Generally, the object species in an isolated, purified or substantially pure composition will comprise more than 80 to 90 percent of all macromolecular species present in a composition. Most preferably, the object species is purified to essential homogeneity (i.e., contaminant species cannot be detected in the composition by conventional detection methods) wherein the composition consists essentially of a single macromolecular species.

### Vaccines

The present invention provides a conformational epitope from streptococcal M protein in a hybrid molecule such that the epitope is provided in a functional conformational state such that it is capable of being immunologically interactive.

By “immunological interactivity” is meant any form of interaction with immune cells or immune effector cells and/or any form of immune response. Generally, immunological interactivity is measured by antibody binding or interactivity with the peptide fragment. However, the immunological interactivity also extends to measuring cellular immune responses.

The present invention further provides a vaccine composition useful against streptococci, the vaccine composition comprising a chimeric peptide according to the invention and one or more pharmaceutically acceptable carriers and/or diluents. The vaccine may further comprise an adjuvant and/or other immune stimulating molecules

The vaccine is of particular use against GAS. However, it is envisaged that vaccines employing the chimeric peptides of the present invention are also useful against other streptococci groups, including Group C streptococci (GCS) and Group G streptococci (GGS).

The present invention also contemplates a vaccine useful in the development of humoral immunity to M protein but minimally cross reactive with heart tissue, the vaccine comprising a chimeric peptide according to the present invention and one or more pharmaceutically acceptable carriers and/or excipients.

The vaccine may contain a single peptide type or a range of peptides covering different or similar epitopes. In addition, or alternatively, a single polypeptide may be provided with multiple epitopes. The latter type of vaccine is referred to as a polyvalent vaccine. A multiple epitope includes two or more repeating epitopes.

The formation of vaccines is generally known in the art and reference can conveniently be made to Remington's Pharmaceutical Sciences, 17th ed., Mack Publishing Co., Easton, Pa., USA.

### Antibodies

The present invention also provides an antibody which binds immunospecifically to a conformational epitope (e.g. B-cell epitope) of a streptococcal, preferably a GAS, M-protein, the conformational epitope comprising at least three amino acids from within SEQ ID NO:1 wherein X¹ is selected from S, E and V; X² is selected from R, N and D; X³ is selected from K and N; and X⁴ is selected from K, R and M, and wherein at least one amino acid of the at least three amino acids is selected from the group consisting of X¹ being E or V, X² being N or D, X³ being N and X⁴ being R or M.

In a preferred embodiment, the antibody binds immunospecifically to a conformational epitope comprising at least three amino acids from within SEQ ID NO:2. wherein X¹ is selected from S, E and V; X² is selected from R, N and D; X³ is selected from K and N; and X⁴ is selected from K, R and M, and wherein at least one amino acid of the at least three amino acids is selected from the group consisting of X¹ being E or V, X² being N or D, X³ being N and X⁴ being R or M.

Preferably, at least one amino acid of the at least three amino acids constituting the conformational epitope is selected from the group consisting of X¹ being E or V, X² being N or D, X³ being N and X⁴ being R or M. In a preferred embodiment, the at least one amino acid is selected from the group consisting of X³ being N and X⁴ being R or M. More preferably, the conformational epitope comprises at least X³ being N and X⁴ being R or M. Yet more preferably, the conformational epitope comprises X¹ being S, X² being R, X³ being N and X⁴ being R or M.

More preferably, the antibody binds immunospecifically to a conformational epitope present within one of the SEQ ID NOs:3-20, yet more preferably to a conformational epitope within SEQ ID NO:19 or 20.

Clearly the antibody will only bind to the epitope when is correctly presented as its conformational epitope, for example as a chimeric peptide according to the present invention.

An antibody is considered to bind immunospecifically to a conformational epitope of a streptococci M-protein if it binds with a lower dissociation constant than existing antibodies. In particular, an antibody is considered to bind immunospecifically to a conformational epitope of a streptococci M-protein as defined above if it binds with a lower dissociation constant than an antibody raised to p145. Preferably, antibodies of the present invention have a dissociation constant (Kd) of less than 10⁻⁶M, more preferably less than 10⁻⁹M, yet more preferably less than 10⁻¹⁰M and yet more preferably less than 10⁻¹²M.

The term “antibody” as used herein and unless the context requires otherwise shall be taken to mean any specific binding substance having a binding domain with the required specificity and/or affinity for an M protein B-cell epitope, including an immunoglobulin, antibody fragment e.g., V_{H}, V_{L}, Fab, Fab′, F(ab)₂, Fv, etc., having binding specificity and/or affinity for an M protein B-cell epitope, or an antibody conjugate comprising such antibodies and/or antibody fragments. The term “antibody” shall also be taken to include a cell expressing an antibody or antibody fragment or antibody conjugate, for example a hybridoma or plasmacytoma expressing a monoclonal antibody or a cell expressing a recombinant antibody fragment or a humanized antibody fragment or a chimeric antibody fragment. Preferred “antibodies” within this definition include intact polyclonal or monoclonal antibodies, an immunoglobulin (IgA, IgD, IgG, IgM, IgE) fraction, a chimeric antibody, a humanized antibody, an antibody fragment, or an immunoglobulin binding domain, whether natural or synthetic, and conjugates comprising same. Chimeric molecules including an immunoglobulin binding domain, or equivalent, fused to another polypeptide are also included within the meaning of the term “antibody” as used herein.

Preferred antibodies, antibody fragments and antibody conjugates are reactive with a conformational epitope of a GAS M-protein and only minimally reactive or non-reactive with a tissue, e.g. the heart tissue, of a subject to whom the antibody is administered.

Preferably, an antibody, antibody fragment or antibody conjugate is produced by a process comprising immunizing an animal with an immunogenic peptide composition comprising a chimeric peptide according to the present invention. Preferably, the immunogenic peptide composition further comprises a carrier protein e.g., diphtheria toxoid (DT) protein, preferably conjugated to the chimeric peptide.

Preferred antibodies are immunoglobulin fractions or monoclonal antibodies or recombinant antibodies or humanized versions thereof.

By “humanized antibody” is meant an antibody, antibody fragment or antibody conjugate comprising variable region framework residues substantially from, for example, a human antibody (termed an acceptor antibody) and complementarity determining regions substantially from, for example, a mouse-antibody, (referred to as the donor immunoglobulin). Constant region(s), if present, is(are) substantially or entirely from a human immunoglobulin. The human variable domains are usually chosen from human antibodies whose framework sequences exhibit a high degree of sequence identity with a murine variable region domain from which the CDR/s were derived. The heavy and light chain variable region framework residues can be derived from the same or different human antibody sequences. The human antibody sequences can be the sequences of naturally-occurring human antibodies or can be consensus sequences of several human antibodies (e.g., as described in WO 92/22653).

The antibody, antibody fragment or antibody conjugate may be of any immunoglobulin isotype e.g., IgM, IgA, IgD, IgE, IgG, including e.g., IgG1, IgG2, etc.

In another example, an antibody conjugate is employed e.g., comprising an antibody or antibody fragment having the desired specificity for an epitope of the streptococcal, and in particular GAS, M-protein conjugated to a toxic agent. The invention clearly extends to any and all such antibody conjugates. Conjugates comprising toxins are particularly useful for targeted cytotoxicity of streptococcal, and in particular GAS, cells. Suitable toxic substances for the production of toxin-containing antibody conjugates will be apparent to the skilled artisan and include, for example, paclitaxol, cytochalasin B, gramicidin D, ethidium bromide, emetine, mitomycin, puromycin and analogs or homologs thereof.

Preferably, the antibodies, antibody fragments and antibody conjugates are modified to enhance their stability in vivo. It will be appreciated by those skilled in the art that a variety of methods may be used to modify the therapeutic antibodies, fragments and conjugates such that their in vivo stability is increased thereby enhancing the effective serum titer of a unit dose. For example, the antibody, antibody fragment or antibody conjugate is PEGylated, and/or the sequence of the immunogenic moiety of a recombinant antibody or antibody fragment is modified to remove one or more protease cleavage sites.

The composition comprising an antibody, antibody fragment or antibody conjugate that binds to an epitope of a streptococcal M-protein as described herein is particularly useful for treating a streptococcal, and in particular GAS, infection or complication thereof in a human or other mammalian subject or for treating a disease associated with streptococcal, and in particular GAS, infection in a human or other mammalian subject. Preferably, the composition is for the treatment of humans.

Without compromising the generality of such compositions for the treatment of immunized and non-immunized subjects alike, the composition comprising an antibody, antibody fragment or antibody conjugate as described according to any embodiment hereof is particularly suited to the prophylactic and/or therapeutic treatment of non-vaccinated or immune-compromized or immune-deficient subjects. By “non-vaccinated” is meant that the subject has not been vaccinated with a peptide-based vaccine comprising an immunogenic peptide derived from a protein of Streptococcus pyogenes. By “immune-compromized” is meant that the subject does not produce endogenous antibody at a level sufficient to prevent the spread or development of streptococcal, and in particular GAS, infection or the progression of disease arising from streptococcal, and in particular GAS, infection as a consequence of infection by another disease agent, radiation damage, treatment (e.g., chemotherapy) or general ill-health, e.g., a subject that is HIV+. By “immune-deficient” is meant that the subject does not have a functional immune system sufficient to produce endogenous antibody at a level to prevent the spread or development of streptococcal, and in particular GAS, infection or the progression of disease arising from streptococcal, and in particular GAS, infection as a consequence of a genetic defect, radiation damage, treatment (e.g., chemotherapy). In immune-compromized and/or immune-deficient subjects, antibodies against a B-cell epitope of, for example, GAS may not be produced at detectable levels, or at a level that reflects bacterial burden. subject exposed to the pathogen.

### Pharmaceutical Compositions

The present invention also provides a pharmaceutical composition comprising one or more antibodies, antibody fragments and antibody conjugates according to the present invention and a pharmaceutically acceptable carrier and/or excipient. Such compositions are formulated without undue experimentation for intravenous, intranasal, intramuscular, oral, subcutaneous, or intradermal delivery, or via suppository or implant (e.g. using slow release molecules). Suitably the pharmaceutical composition comprises a unit dose of one or more antibodies, antibody fragments and antibody and a pharmaceutically acceptable carrier or excipient. Preferred unit doses of antibody, antibody fragment or antibody conjugate generally comprise from about 0.1 μg immunoglobulin per kilogram body weight to about 100 mg immunoglobulin per kilogram body weight, preferably from about 0.1 μg immunoglobulin per kilogram body weight to about 20 mg immunoglobulin per kilogram body weight, more preferably from about 0.1 μg immunoglobulin per kilogram body weight to about 10 mg immunoglobulin per kilogram body weight, and still more preferably from about 0.1 μg immunoglobulin per kilogram body weight to about 1.0 mg immunoglobulin per kilogram body weight. Suitable carriers and excipients will vary according to the mode of administration and storage requirements of a composition comprising an antibody, antibody fragment or antibody conjugate and are described herein.

The active ingredients of a pharmaceutical composition comprising an antibody are contemplated herein to exhibit excellent therapeutic activity, for example, in the passive transfer of therapeutic antibodies to M protein of streptococci but the antibodies being only minimally reactive with heart tissue when administered in amount which depends on the particular case. Dosage regime may be adjusted to provide the optimum therapeutic response. For example, several divided doses may be administered daily or the dose may be proportionally reduced as indicated by the exigencies of the therapeutic situation. The active compound may be administered in a convenient manner such as by the oral, intravenous (where water soluble), intramuscular, subcutaneous, intranasal, intradermal or suppository routes or implanting (e.g. using slow release molecules). Depending on the route of administration, the active ingredients which comprise an antibody, antibody fragment or antibody conjugate may be required to be coated in a material to protect the ingredients from the action of enzymes, acids and other natural conditions which may inactivate the ingredients.

The active compounds may also be administered parenterally or intraperitoneally. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof and vegetable oils. The proper fluidity can be maintained, for example, by the use of a coating such as lecithin, by the maintenance of the required particle size in the case of dispersion and by the use of surfactants. The preventions of the action of microorganisms can be brought about by various antibacterial and antifungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

Sustained release injectable formulations are produced e.g., by encapsulating the antibody in porous microparticles which comprise a pharmaceutical agent and a matrix material having a volume average diameter between about 1 μm and 150 μm, e.g., between about 5 μm and 25 μm diameter. In one embodiment, the porous microparticles have an average porosity between about 5% and 90% by volume. In one embodiment, the porous microparticles further comprise one or more surfactants, such as a phospholipid. The microparticles may be dispersed in a pharmaceutically acceptable aqueous or non-aqueous vehicle for injection. Suitable matrix materials for such formulations comprise a biocompatible synthetic polymer, a lipid, a hydrophobic molecule, or a combination thereof. For example, the synthetic polymer can comprise, for example, a polymer selected from the group consisting of poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers, derivatives and blends thereof. In a preferred embodiment, the synthetic polymer comprises a poly(lactic acid), a poly(glycolic acid), a poly(lactic-co-glycolic acid), or a poly(lactide-co-glycolide).

When the therapeutic antibodies are suitably protected as described above, the active, compound may be orally administered, for example, with an inert diluent or with an assimilatable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 ug and 2000 mg of active compound.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

The present invention provides a composition comprising an amount of a nucleic acid encoding an antibody or antibody fragment described according to any embodiment hereof that is sufficient to treat and/or prevent streptococcal infection, and in particular GAS infection, or complication thereof in a subject or a disease or complication associated with streptococcal infection, and in particular GAS infection, in a subject wherein the antibody or fragment binds immunospecifically to a conformational epitope (e.g. a B-cell epitope) of a streptococcal, and in particular GAS, M-protein.

Preferably, the nucleic acid is operably-linked to a promoter that induces expression of the antibody or antibody fragment in a cell, tissue or organ of a subject to whom it is administered. For example, in the case of a nucleic acid for administration to a human subject, the nucleic acid encoding the antibody or antibody fragment is operably linked to a promoter capable of inducing expression of the antibody or fragment in a human cell, tissue or organ. Suitable promoters will be apparent to the skilled artisan and include for example, an immediate early promoter from human cytomegalovirus or a SV40 promoter.

The present invention also provides an isolated cell expressing an antibody, antibody fragment or antibody conjugate as described according to any embodiment hereof e.g., example, a hybridoma or plasmacytoma expressing the antibody or antibody fragment or a cell expressing a recombinant antibody or recombinant antibody fragment, or a conjugate comprising such antibodies or antibody fragments.

The present invention also provides a composition comprising an amount of one or more cells expressing an antibody, antibody fragment or antibody conjugate sufficient to treat and/or prevent streptococcal infection, and in particular GAS infection, or complication thereof in a subject or a disease or complication associated with streptococcal, and in particular GAS, infection in a subject wherein the antibody, antibody fragment or antibody conjugate binds immunospecifically to a conformational epitope (e.g. a B-cell epitope) of a streptococcal, and in particular a GAS, M-protein.

Preferably, the cell is a cell expressing a recombinant antibody or a fragment thereof or a conjugate comprising the antibody or fragment. For example, the cell is a cell from a subject to be treated, e.g., a blood cell from a subject, e.g., a leukocyte cell from a subject.

The present invention also provides a method of treating or ameliorating streptococcal, and in particular GAS, infection in a human or other mammalian subject the method comprising administering to the subject a composition as described according to any embodiment hereof, wherein the composition is administered in an amount effective to prevent an increase in bacterial count or to reduce bacterial count in a sample from the subject.

In an alternative embodiment, the present invention also provides a method of preventing, ameliorating or treating a disease or complication associated with streptococcal, and in particular GAS, infection of a human or other mammalian subject the method comprising administering to the subject a composition as described according to any embodiment hereof, wherein the composition is administered in an amount effective to reduce the severity of one or more disease symptoms or to prevent onset of one or more diseases arising from GAS infection.

In a further alternative embodiment, the present invention also provides a method of neutralizing a streptococcal, and in particular GAS, pathogen in a subject exposed to the pathogen the method comprising administering to a subject infected with the streptococcal, and in particular GAS, pathogen a composition as described according to any embodiment hereof, wherein the composition is administered in an amount effective to opsonize the pathogen in the serum of the subject. The term “opsonize” as used herein should be construed as promoting the ingestion and killing of GAS by phagocytic cells in the subject.

Preferably, the composition is administered for a time and under conditions sufficient to achieve the stated purpose, e.g., to prevent an increase in bacterial count or to reduce bacterial count in a sample from the subject or to reduce the severity of one or more disease symptoms or to prevent onset of one or more diseases arising from the streptococcal, and in particular GAS, infection or to opsonize a streptococcal, and in particular GAS, pathogen in the serum of the subject. For example, the composition is administered by continuous infusion, or is administered a plurality of times to thereby achieve the stated purpose.

Preferred diseases or complications associated with streptococcal infection, and in particular GAS infection, in the present context include, but are not limited to, uncomplicated pharyngitis, pyoderma, skin infection, soft tissue infection, bacteremia, necrotizing fasciitis, rheumatic fever, rheumatic heart disease, acute glomerulonephritis, or morbidity, amongst others.

Preferably, the pharmaceutical composition is co-administered with an antibiotic having bacteriostatic or bacteriocidal activity against streptococci infection, e.g. the S. pyogenes (GAS) infection. By “co-administered” is meant that the antibiotic is administered to the subject for the purposes of treating the same infection as the pharmaceutical composition of the invention, irrespective of whether or not antibiotic and pharmaceutical composition are administered in the same or a different unit dose or at the same or a different time. Generally, albeit not necessarily, the antibiotic and pharmaceutical composition will be administered in different unit doses. Such compounds are administered by well-established routes, generally orally or by intravenous or intramuscular injection. In a particularly preferred embodiment, the antibiotic is a penicillin compound e.g., amoxicillin, erythromycin, cephalexin, cefadroxil, cefaclor, cefuroxime axatil, cefizime, cefdinir, penicillin VK, penicillin G benzathine, or a mixture thereof. The co-administration of other antibiotics is not to be excluded.

The subject is preferably a non-vaccinated or immune-compromized or immune-deficient subject. Alternatively, or in addition, the subject is an individual that has been vaccinated against a strain of streptococci, and in particular S. pyogenes, wherein the vaccination has not resulted in protection sufficient to prevent a subsequent infection or to prevent the onset of disease associated with infection thereby. Alternatively, or in addition, the subject is an individual that has been vaccinated against a strain of streptococci, and in particular S. pyogenes, however is immune-compromized or immune-deficient.

Preferably, the subject is human.

In use, the antibody, antibody fragment or antibody conjugate is preferably bacteriostatic or bacteriocidal. Preferably, the antibody, antibody fragment or antibody conjugate is bacteriocidal. Such bactericidal activity can be conferred by a toxic agent conjugated to an antibody, antibody fragment or antibody conjugate. Alternatively or in addition, the antibody, antibody fragment or antibody conjugate can have bactericidal activity without the need for a toxin conjugate, e.g., by inducing antibody dependent cell cytotoxicity.

The present invention also provides a method of maintaining a therapeutically or prophylactically effective serum titer of an antibody against a streptococcal M protein, and in particular an M-protein of S. pyogenes (GAS), in a subject the method comprising administering a plurality of doses of a composition as described according to any embodiment hereof, wherein the each of the doses is administered in an amount effective to prevent an increase in bacterial count or to reduce bacterial count in a sample from the subject and/or to reduce the severity of one or more disease symptoms or to prevent onset of one or more diseases arising from a streptococcal, and in particular a GAS, infection, and/or to opsonize the pathogen in the serum of the subject. Preferably, the method further comprises monitoring antibody titre in the serum of the subject to thereby determine when antibody titres are declining in the serum. Preferably, second and subsequent doses of the composition are administered when antibody titres in serum are decreasing e.g., after antibody titres commence their decline and/or before they have reached a minimum level in the serum.

In one example, a method as described according to any embodiment hereof additionally comprises providing or obtaining a composition as described according to any embodiment hereof or information concerning same. For example, the present invention provides a method of treating or ameliorating a streptococcal, and in particular a GAS, infection in a human or other mammalian subject or preventing, ameliorating or treating a disease or complication associated with streptococcal, and in particular GAS, infection of a human or other mammalian subject or neutralizing a streptococcal, and in particular a GAS, infection in a subject exposed to the pathogen, the method comprising:

(i) determining a subject suffering from a streptococcal, and in particular a GAS, infection or a disease or complication associated with streptococcal, and in particular GAS, infection or at risk of developing a streptococcal, and in particular a GAS, infection or a disease or complication associated with streptococcal, and in particular GAS, infection;
(ii) obtaining a composition as described according to any embodiment hereof; and
(iii) administering the composition to the subject.

In another example, a method of treating or ameliorating streptococcal, and in particular GAS, infection in a human or other mammalian subject or preventing, ameliorating or treating a disease or complication associated with streptococcal infection, and in particular GAS infection, of a human or other mammalian subject or neutralizing a streptococcal, and in particular a GAS, pathogen in a subject exposed to the pathogen, the method comprising:

(i) identifying a subject suffering from a streptococcal infection, and in particular a GAS infection, or a disease or complication associated with streptococcal infection, and in particular GAS infection, or at risk of developing a streptococcal infection, and in particular a GAS infection, or a disease or complication associated with streptococcal infection, and in particular GAS infection; and
(ii) recommending administration of a composition as described according to any embodiment hereof.

Alternatively, the present invention provides a method of treating or ameliorating streptococcal, and in particular GAS, infection in a human or other mammalian subject or preventing, ameliorating or treating a disease or complication associated with streptococcal, and in particular GAS, infection of a human or other mammalian subject or neutralizing a streptococcal, and in particular a GAS, pathogen in a subject exposed to the pathogen the method comprising administering or recommending administration of a composition as described according to any embodiment hereof to a subject previously identified as suffering from a streptococcal, and in particular a GAS, infection or a disease or complication associated with streptococcal, and in particular GAS, infection or at risk of developing a streptococcal, and in particular a GAS, infection or a disease or complication associated with streptococcal, and in particular GAS, infection.

The present invention also provides an amount of an antibody, antibody fragment or antibody conjugate that binds immunospecifically to a conformational epitope (e.g. a B-cell epitope) of a streptococcal, and in particular a GAS, M-protein, or nucleic acid encoding the antibody, antibody fragment or antibody conjugate, or a cell expressing the antibody, antibody fragment or antibody conjugate, or a composition comprising the antibody, antibody fragment, antibody conjugate, nucleic acid, or cell, for use in medicine e.g., for therapy of an acute severe streptococcal, and in particular a GAS, infection or a disease or complication associated therewith, such as uncomplicated pharyngitis, pyoderma, skin infection, soft tissue infection, bacteremia, necrotizing fasciitis, rheumatic fever, rheumatic heart disease, acute glomerulonephritis, or morbidity.

The present invention also provides an amount of an antibody, antibody fragment or antibody conjugate that binds immunospecifically to a conformational epitope (e.g. a B-cell epitope) of a streptococcal, and in particular a GAS, M-protein, or nucleic acid encoding the antibody, antibody fragment or antibody conjugate, or a cell expressing the antibody, antibody fragment or antibody conjugate, in the preparation or manufacture of a medicament for the treatment of an acute severe streptococcal, and in particular GAS, infection or a disease or complication associated therewith, such as uncomplicated pharyngitis, pyoderma, skin infection, soft tissue infection, bacteremia, necrotizing fasciitis, rheumatic fever, rheumatic heart disease, acute glomerulonephritis, or morbidity. In one example, the method further comprises establishing a correlation between affinity of an antibody, antibody fragment or antibody conjugate for an epitope of an M-protein of GAS and a desired bioactivity e.g., activity against GAS cell viability or growth or cell division. By virtue of establishing such a correlation, it is also possible to then perform a modification of this method by:

(i) producing, isolating or obtaining an antibody, antibody fragment or antibody conjugate that binds to a B-cell epitope of an M-protein of GAS; and
(ii) isolating an antibody, antibody fragment or antibody conjugate from (i) that binds at a desired affinity to a B-cell epitope of an M-protein of GAS, thereby isolating an antibody for treating or ameliorating GAS infection in a human or other mammalian subject or for preventing, ameliorating or treating a disease or complication associated with GAS infection of a human or other mammalian subject or for neutralizing a GAS pathogen in a

As used herein, the term “treat” or variations thereof such as “treatment” shall be taken to mean a treatment following streptococcal infection, and in particular GAS infection, that results in reduced bacterial count, prevention or reduction in severity of one or more symptoms of streptococcal, and in particular GAS, infection, e.g., uncomplicated pharyngitis, pyoderma, skin infection, soft tissue infection, bacteremia, necrotizing fasciitis, rheumatic fever, rheumatic heart disease, acute glomerulonephritis or morbidity, amongst others. It is to be understood that such treatment therefore includes the prophylaxis of streptococcal, and in particular GAS, infection insofar as it prevents or reduces symptom development in an infected individual and/or prevents development of a complication thereof. Alternatively, or in addition, treatment in the present context also includes the prophylaxis of streptococcal, and in particular GAS, infection insofar as it prevents or reduces an increase in bacterial load in an infected individual.

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is known in the art. Except insofar as any conventional media or agent is incompatible with the active ingredient, use thereof in the therapeutic compositions is contemplated. Supplementary active ingredients can also be incorporated into the compositions.

It is advantageous to formulate parenteral compositions in dosage unit form for ease of administration and uniformity of dosage. “Dosage unit form” as used herein refers to physically discrete units suited as unitary dosages for the mammalian subjects to be treated; each unit containing a predetermined quantity of active material calculated to produce the desired therapeutic effect in association with the required pharmaceutical carrier. The specification for the dosage unit forms of a pharmaceutical composition of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active ingredient is compounded for convenient and effective administration in effective amounts with a suitable pharmaceutically acceptable carrier in dosage unit form as hereinbefore disclosed. A unit dosage form can, for example, contain the principal active compound in an amount ranging from about 0.1 μg to about 100 mg. In the case of compositions containing supplementary active ingredients, the dosages are determined by reference to the usual dose and manner of administration of the ingredients.

### Additional Components in Pharmaceutical Compositions

In another example, an antibody, antibody fragment or antibody conjugate that binds to an epitope of an M-protein of GAS is formulated in combination with another antimicrobial agent or antibiotic active against GAS. Combinations of the antibody, antibody fragment, antibody conjugate and other agents are useful to allow antibiotics to be used at lower doses due to toxicity concerns, to enhance the activity of antibiotics whose efficacy has been reduced or to effectuate a synergism between the components such that the combination is more effective than the sum of the efficacy of either component independently. Antibiotics that may be combined with the antibody, antibody fragment or antibody conjugate include but are not limited to penicillin, ampicillin, amoxycillin, vancomycin, cycloserine, bacitracin, cephalolsporin, methicillin, streptomycin, kanamycin, tobramycin, gentamicin, tetracycline, chlortetracycline, doxycycline, chloramphenicol, lincomycin, clindamycin, erythromycin, oleandomycin, polymyxin nalidixic acid, rifamycin, rifampicin, gantrisin, trimethoprim, isoniazid, paraminosalicylic acid, and ethambutol.

As used herein, the term “pharmaceutically acceptable carrier and/or excipient” shall be taken to mean a compound or mixture thereof that is suitable for use in a composition for administration to a subject for the treatment of streptococcal, and in particular GAS, infection or complication thereof in a subject or a disease or complication associated with streptococcal, and in particular GAS, infection in a subject or for vaccination of a subject against a streptococcal, and in particular a GAS, infection. For example, a suitable carrier or excipient for use in a pharmaceutical composition for injection into a subject will generally not cause an adverse response in a subject.

A carrier or excipient useful in a vaccine or pharmaceutical composition will generally not inhibit to any significant degree a relevant biological activity of an antibody, antibody fragment, antibody conjugate or chimeric peptide as described according to any embodiment hereof e.g., the carrier or excipient will not significantly inhibit the ability of an antibody, fragment or conjugate to bind to an streptococcal, and in particular GAS, M protein and/or to prevent the growth or spread of streptococcal, and in particular GAS, cells and/or to kill streptococcal, and in particular GAS, cells, or significantly inhibit the ability of a chimeric peptide to elicit an antibody response. For example, a carrier or excipient may merely provide a buffering activity to maintain the active compound at a suitable pH to thereby exert its biological activity, e.g., phosphate buffered saline. Alternatively, or in addition, the carrier or excipient may comprise a compound that enhances the activity or half-life of the chimeric peptide, antibody, antibody fragment or antibody conjugate, e.g., a protease inhibitor. In yet another example, the carrier or excipient may include an antibiotic and/or an anti-inflammatory compound.

It will be appreciated by those skilled in the art that the invention also encompasses sustained release compositions comprising one or more chimeric antibodies, antibodies, antibody fragments or antibody conjugates as described according to any embodiment hereof, e.g., to reduce the dosage required and/or frequency of administration to a subject and/or to prolong serum titer following administration.

As used herein, the term “amount effective” refers to the amount of a therapeutic composition (e.g. a pharmaceutical compositions) comprising an antibody, antibody fragment or antibody conjugate that binds to an epitope of a strepotococcal, and in particular a GAS, M-protein, is sufficient to reduce the severity, and/or duration of a streptococcal, and in particular a GAS, infection; ameliorate one or more symptoms thereof, prevent the advancement of a streptococcal, and in particular a GAS, infection or cause regression of a streptococcal, and in particular a GAS, infection or which is sufficient to result in the prevention of the development, recurrence, onset, or progression of a streptococcal infection, and in particular a GAS infection, or one or more symptoms thereof, or enhance or improve the prophylactic and/or therapeutic effect(s) of another therapy (e.g., another therapeutic agent).

The efficacy of treatment is established by any means known to the skilled artisan e.g., by determining live cell count in a sample from the subject such as, for example, serology based on cultures from clinical specimens such as sera or throat swab. For example, serologic methods can detect group A antigen; or by the precipitin test. Alternatively, or in addition the efficacy of treatment is determined by determining bacitracin sensitivity of a clinical specimen. Bacitracin sensitivity presumptively differentiates group A from other b-hemolytic streptococci (B, C, G). Alternatively, or in addition, acute glomerulonephritis and acute rheumatic fever are identified by anti-streptococcal antibody titres in serum from a subject. In addition, diseases associated with GAS infection such as acute rheumatic fever are diagnosed by clinical criteria.

Therapy of GAS and/or Complications Thereof.

The compositions described according to any embodiment hereof may be administered to a subject to prevent severe GAS infection in a subject.

Accordingly, the present invention also encompasses methods for achieving a serum titre of at least about 40 μg/ml of one or more antibodies or fragments thereof that immunospecifically bind to one or more B-cell epitopes in a mammal, preferably a primate and most preferably a human. For example, the present invention provides methods for achieving a serum titer of at least about 40 μg/ml (preferably at least about 75 μg/ml, more preferably at least about 100 μg/ml, and most preferably at least about 150 μg/ml) of an antibody or fragment thereof that immunospecifically binds to a B-cell epitopes of M-protein GAS in a mammal, comprising administering a dose of less than 2.5 mg/kg (preferably 1.5 mg/kg or less) of the antibody to the non-primate mammal and measuring the serum titer of the antibody or antibody fragment at least 1 day after administering the dose to the mammal. The present invention also provides methods for achieving a serum titer of at least about 150 μg/ml (preferably at least about 200 μg/ml) of an antibody or fragment thereof that immunospecifically binds to a B-cell epitopes of M-protein GAS in a mammal, comprising administering a dose of approximately 5 mg/kg of the antibody or antibody fragment to the mammal and measuring the serum titer of the antibody or antibody fragment at least 1 day after the administration of the dose to the mammal.

The present invention also provides methods for achieving a serum titer of at about least 40 μg/ml of an antibody or fragment thereof that immunospecifically binds to a B-cell epitopes of M-protein GAS in a primate, comprising administering a first dose of 10 mg/kg (preferably 5 mg/kg or less and more preferably 1.5 mg/kg or less) of the antibody or antibody fragment to the primate and measuring the serum titer of the antibody or antibody fragment 20 days (preferably 25, 30, 35 or 40 days) after administrating the first dose to the primate and prior to the administration of any subsequent dose. The present invention also provides methods for achieving a serum titer of at least about 75 μg/ml (preferably at least about 100 μg/ml, at least about 150 μg/ml, or at least about 200 μg/ml) of an antibody or fragment thereof that immunospecifically binds to a B-cell epitopes of M-protein GAS in a primate, comprising administering a first dose of approximately 15 mg/kg of the antibody or antibody fragment to the primate and measuring the serum titer of the antibody or antibody fragment 20 days (preferably 25, 30, 35 or 40 days) after administering the first dose to the primate but prior to any subsequent dose.

The present invention also provides methods for preventing, treating, or ameliorating one or more symptoms associated with a GAS infection in a human subject, the methods comprising administering to the human subject at least a first dose of approximately 15 mg/kg of an antibody or fragment thereof that immunospecifically binds to a B-cell epitopes of M-protein GAS so that the human subject has a serum antibody titer of at least about 75 μg/ml, preferably at least about 100 μg/ml, at least about 150 μg/ml, or at least about 200 μg/ml 30 days after the administration of the first dose of the antibody or antibody fragment and prior to the administration of a subsequent dose. The present invention also provides methods for preventing, treating or ameliorating one or more symptoms associated with a GAS infection in a human subject, the methods comprising administering to the human subject at least a first dose of less than 15 mg/kg (preferably 10 mg/kg or less, more preferably 5 mg/kg or less, and most preferably 1.5 mg/kg or less) of an antibody or fragment thereof that immunospecifically binds to a B-cell epitopes of M-protein GAS so that the human subject has a serum antibody titer of at least about 75 μg/ml, preferably at least about 100 μg/ml, at least about 150 μg/ml, or at least about 200 μg/ml 30 days after the administration of the first dose of the antibody or antibody fragment and prior to the administration of a subsequent dose. The present invention further provides methods for preventing, treating or ameliorating one or more symptoms associated with a GAS infection in a human subject, the methods comprising administering to the human subject a first dose of an antibody or fragment thereof that immunospecifically binds to a B-cell epitopes of M-protein GAS such that a prophylactically or therapeutically effective serum titer of less than about 10 μg/ml is achieved no more than 30 days after administering the antibody or antibody fragment.

### Diagnostics

The chimeric peptides can be used to screen for naturally occurring antibodies to M protein. Alternatively, specific antibodies can be used to screen for M protein. Techniques for such assays are well known in the art and include, for example, sandwich assays and ELISA.

In accordance with this aspect of the present invention, the chimeric peptides are particularly useful in screening for antibodies to M protein and, hence, provide a diagnostic protocol for detecting streptococcal infection. Alternatively, biological samples, such as blood serum, sputum, tissue and tissue extracts can be directly screened for M protein using antibodies raised to the chimeric peptides.

Accordingly, there is provided a method for the diagnosis of streptococcal infection in a subject comprising contacting a biological sample from the subject with an antibody binding effective amount of a chimeric peptide for a time and under conditions sufficient for an antibody-chimeric peptide complex to form, and then detecting the complex.

The presence of M protein antibodies in a patient's blood serum, tissue, tissue extract or other bodily fluid, can be detected using a wide range of immunoassay techniques such as those described in U.S. Pat. Nos. 4,016,043, 4,424,279 and 4,018,653. This includes both single-site and two-site, or “sandwich”, assays of the non-competitive types, as well as in the traditional competitive binding assays. Sandwich assays are among the most useful and commonly used assays and are favoured for use in the present invention. A number of variations of the sandwich assay technique exist, and all are intended to be encompassed by the present invention. Briefly, in a typical forward assay, a chimeric peptide is immobilised onto a solid substrate to form a first complex and the sample to be tested for M protein antibody brought into contact with the bound molecule. After a suitable period of incubation, for a period of time sufficient to allow formation of an chimeric-peptide-antibody secondary complex. An anti-immunoglobulin antibody, labelled with a reporter molecule capable of producing a detectable signal, is then added and incubated, allowing time sufficient for the formation of a tertiary complex of chimeric peptide-antibody-labelled antibody. Any unreacted material is washed away, and the presence of the first antibody is determined by observation of a signal produced by the reporter molecule. The results may either be qualitative, by simple observation of the visible signal or may be quantitated by comparing with a control sample containing known amounts of hapten. Variations of the forward assay include a simultaneous assay, in which both sample and labelled antibody are added simultaneously to the bound antibody, or a reverse assay in which the labelled antibody and sample to be tested are first combined, incubated and then added simultaneously to the bound antibody. These techniques are well known to those skilled in the art, and the possibility of minor variations will be readily apparent. A similar approach is adopted to detect M protein. The antibodies used above may be monoclonal or polyclonal.

The solid substrate is typically glass or a polymer, the most commonly used polymers being cellulose, polyacrylamide, nylon, polystyrene, polyvinyl chloride or polypropylene. The solid supports may be in the form of tubes, beads, discs or microplates, or any other surface suitable for conducting an immunoassay. The binding processes are well-known in the art and generally consist of cross-linking covalently binding or physically adsorbing the molecule to the insoluble carrier.

By “reporter molecule”, as used in the present specification, is meant a molecule which, by its chemical nature, produces an analytically identifiable signal which allows the detection of antigen-bound antibody. Detection may be either qualitative or quantitative. The most commonly used reporter molecule in this type of assay re either enzymes, fluorophores or radionuclide containing molecules (i.e. radioisotopes). In the case of an enzyme immunoassay, an enzyme is conjugated to the second antibody, generally by means of glutaraldehyde or periodate. As will be readily recognised, however, a wide variety of different conjugation techniques exist which are readily available to one skilled in the art. Commonly used enzymes include horseradish peroxidase, glucose oxidase, □-galactosidase and alkaline phosphatase, amongst others. The substrates to be used with the specific enzymes are generally chosen for the production, upon hydrolysis by the corresponding enzyme, of a detectable colour change. It is also possible to employ fluorogenic substrates, which yield a fluorescent product.

Alternatively, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state of excitability in the molecule, followed by emission of the light at a characteristic colour visually detectable with a light microscope. As in the EIA, the fluorescent labelled antibody is allowed to bind to the first antibody-hapten complex. After washing off the unbound reagent, the remaining ternary complex is then exposed to the light of the appropriate wavelength, the fluorescence observed indicates the presence of the hapten of interest. Immunofluorescence and EIA techniques are both very well established in the art and are particularly preferred for the present method. However, other reporter molecules, such as radioisotope, chemiluminescent or bioluminescent molecules, may also be employed. It will be readily apparent to the skilled technician how to vary the procedure to suit the required purpose. It will also be apparent that the foregoing can be used to label chimeric peptides and to use same directly in the detection of M protein antibodies.

The present invention will now be illustrated by the following examples, which are not intended to be limiting in any way. The teachings of all references cited herein are incorporated herein by reference.

### Example 1

### Design of Peptide Library

Amino acids in the p145 protein were changed based on their relative occurrence at each position in the α-helical structure as follows. This resulted in the identification of following peptides:

| Peptide # | N-terminal | 1 (d) | 2 (e) | 3 (f) | 4 (g) | 5 (a) | 6 (b) | 7 (c) | 8 (d) | 9 (e) | 10 (f) | 11 (g) | 12 (a) | C-terminal |
|---|---|---|---|---|---|---|---|---|---|---|---|---|---|---|
| 1 (p145) | LRRDLDA | S | R | E | A | K | K | Q | V | E | K | A | L | E |
| 6 | LRRDLDA | E | R | E | A | K | K | Q | V | E | K | A | L | E |
| 7 | LRRDLDA | V | R | E | A | K | K | Q | V | E | K | A | L | E |
| 12 | LRRDLDA | S | N | E | A | K | K | Q | V | E | K | A | L | E |
| 14 | LRRDLDA | S | D | E | A | K | K | Q | V | E | K | A | L | E |
| 41 | LRRDLDA | S | R | E | A | K | N | Q | V | E | K | A | L | E |
| 67 | LRRDLDA | S | R | E | A | K | K | Q | V | E | R | A | L | E |
| 73 | LRRDLDA | S | R | E | A | K | K | Q | V | E | M | A | L | E |

These peptides were then used as the basis for designing 19 new peptides, each with 2 amino acid changes as below (designated TN 1-19).

### Example 2

### Double Mutant (TN) Peptides for Testing

The following 19 peptides (TN 1-19) were synthesised.

| Peptide # | N-terminal | 1 (d) | 2 (e) | 3 (f) | 4 (g) | 5 (a) | 6 (b) | 7 (c) | 8 (d) | 9 (e) | 10 (f) | 11 (g) | 12 (a) | C-terminal |
|---|---|---|---|---|---|---|---|---|---|---|---|---|---|---|
| 1 | LRRDLDA | S | R | E | A | K | K | Q | V | E | K | A | L | EC |
| 2 | LRRDLDA | E | N | E | A | K | K | Q | V | E | K | A | L | EC |
| 3 | LRRDLDA | E | D | E | A | K | K | Q | V | E | K | A | L | EC |
| 4 | LRRDLDA | E | R | E | A | K | N | Q | V | E | K | A | L | EC |
| 5 | LRRDLDA | E | R | E | A | K | K | Q | V | E | R | A | L | EC |
| 6 | LRRDLDA | E | R | E | A | K | K | Q | V | E | M | A | L | EC |
| 7 | LRRDLDA | V | N | E | A | K | K | Q | V | E | K | A | L | EC |
| 8 | LRRDLDA | V | D | E | A | K | K | Q | V | E | K | A | L | EC |
| 9 | LRRDLDA | V | R | E | A | K | N | Q | V | E | K | A | L | EC |
| 10 | LRRDLDA | V | R | E | A | K | K | Q | V | E | R | A | L | EC |
| 11 | LRRDLDA | V | R | E | A | K | K | Q | V | E | M | A | L | EC |
| 12 | LRRDLDA | S | N | E | A | K | N | Q | V | E | K | A | L | EC |
| 13 | LRRDLDA | S | N | E | A | K | K | Q | V | E | R | A | L | EC |
| 14 | LRRDLDA | S | N | E | A | K | K | Q | V | E | M | A | L | EC |
| 15 | LRRDLDA | S | D | E | A | K | N | Q | V | E | K | A | L | EC |
| 16 | LRRDLDA | S | D | E | A | K | K | Q | V | E | R | A | L | EC |
| 17 | LRRDLDA | S | D | E | A | K | K | Q | V | E | M | A | L | EC |
| 18 | LRRDLDA | S | R | E | A | K | N | Q | V | E | R | A | L | EC |
| 19 | LRRDLDA | S | R | E | A | K | N | Q | V | E | M | A | L | EC |

### Example 3

### Reactivity of p145 Derived Peptide Antisera Against TN 1-19 Peptides

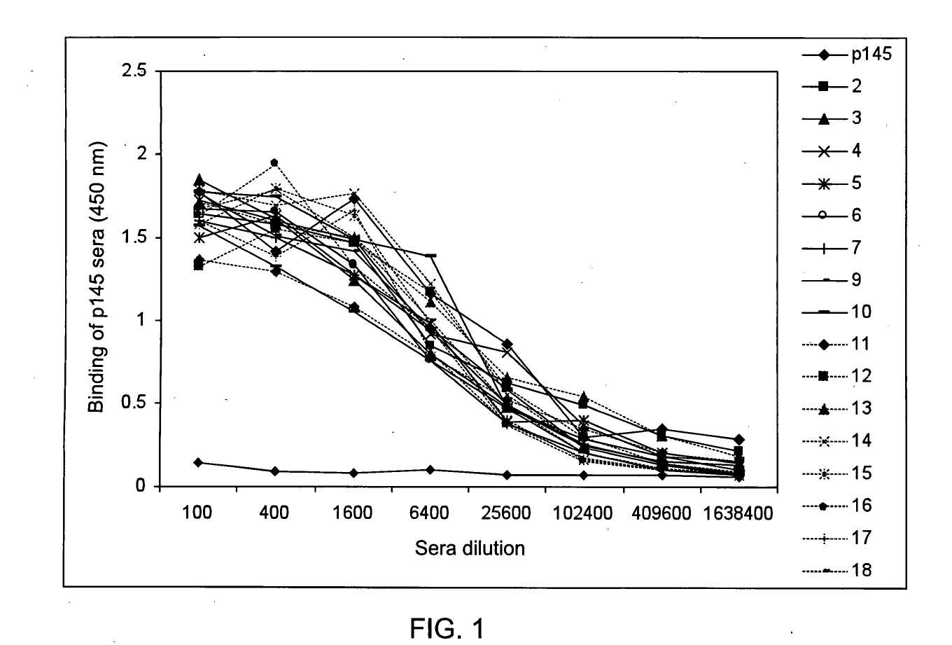

The protocols for ELISA have been previously described (Pruksakom et al 1992; 1994a and WO 96/11944). TN peptides 1-19 were coated at a concentration of 0.5 Tg/ml. All reactions were developed with OPD substrate kit (Sigma Chemical Co) and the absorbance read at 450 nm. The binding affinities are presented in FIG. 1. All 19 TN peptides demonstrated good affinity for p 145 antisera.

### Example 4

### Production of Murine Antisera

Quackenbush mice were immunised subcutaneously in the base of the tail (Pruksakorn et al, 1992). Mice were pre-bled at −1 days. Mice were immunised with 30 μg peptide dissolved in PBS and emulsified in complete Freund's adjuvant. Mice were bled at 20, 27, and 45 days (bleeds 1, 2, 3 and final (fullbleed), respectively). The mice received boosts in PBS at 21 and 28 days (boosts 1 and 2, respectively). In the case of non-responding peptides, mice received a third boost in incomplete Freund's adjuvant (boost 3) at 36 days.

### Example 5

### Peptide Specific IgG Titres

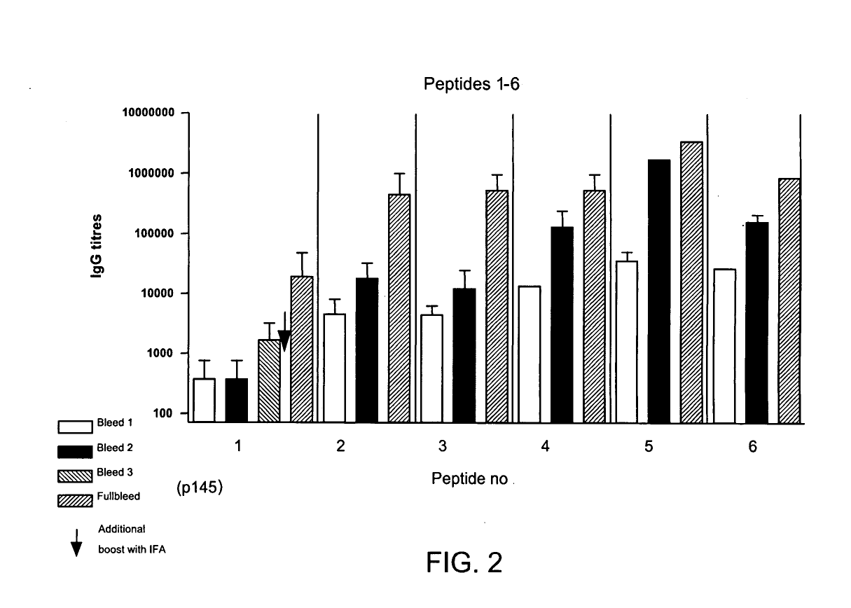

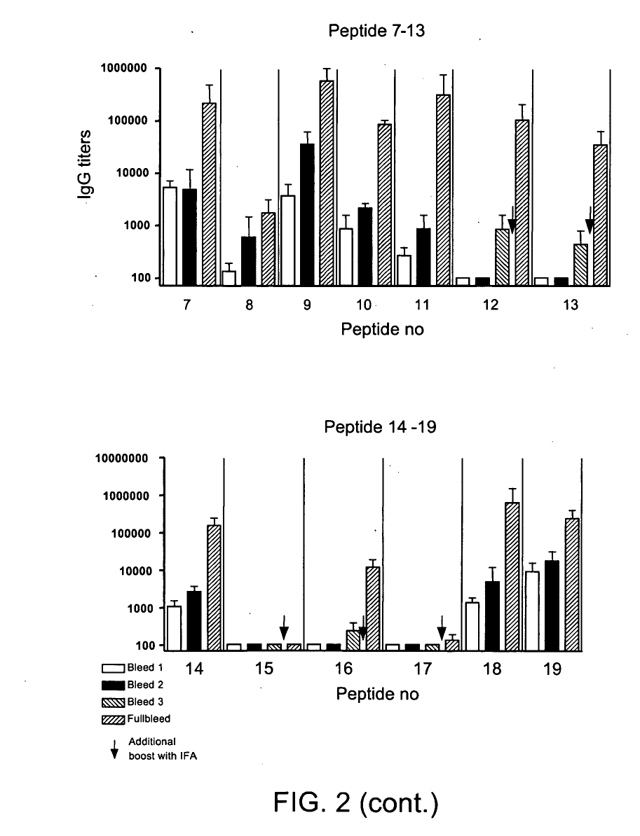

The peptide specific IgG titres in the murine sera samples were analysed. The results are presented in FIG. 2. TN peptides 2, 3, 4, 5, 6, 7, 10, 11, 12, 14, 18 and 19 all gave titres in excess of 100000. TN peptides 3, 4, 5, 6, 9 and 18 all gave titres in excess of 500000

### Example 6

### Purification of Peptide Specific Antibodies

Antibodies to TN peptides 1-19 were affinity purified from the mouse antisera using a column displaying multiple copies of the peptides. The conjugation efficiency of the TN peptides for the purified antibodies are presented in Table 1.

TABLE 1
Conjugation efficiency of the TN peptides
for the purified antibodies

| Peptide # | % |
|---|---|
| 1 | 92.7 |
| 2 | 100 |
| 3 | 76.7 |
| 4 | 96 |
| 5 | 92 |
| 6 | 100 |
| 7 | 98 |
| 8 | 100 |
| 9 | 99 |
| 10 | 98 |
| 11 | 100 |
| 12 | 100 |
| 13 | 100 |
| 14 | 100 |
| 16 | 100 |
| 18 | 100 |
| 19 | 55 |
| anti mouse Ig | 46.3 |

### Example 7

### Binding of Purified Anti-TN Peptide Antibodies to p145

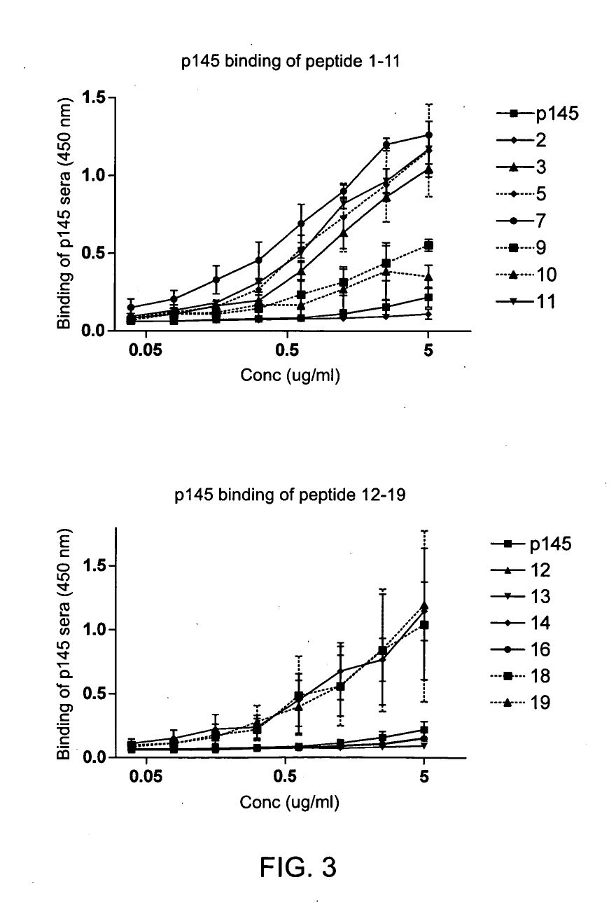

The purified anti-TN peptide antibodies were tested for their ability to bind to p145. The results are presented in FIG. 3. The antibodies with the highest binding affinities for p145 were nos. 3, 5, 7, 11, 14, 18 and 19.

### Example 8

### Binding of Purified Anti-TN Peptide Antibodies to GAS

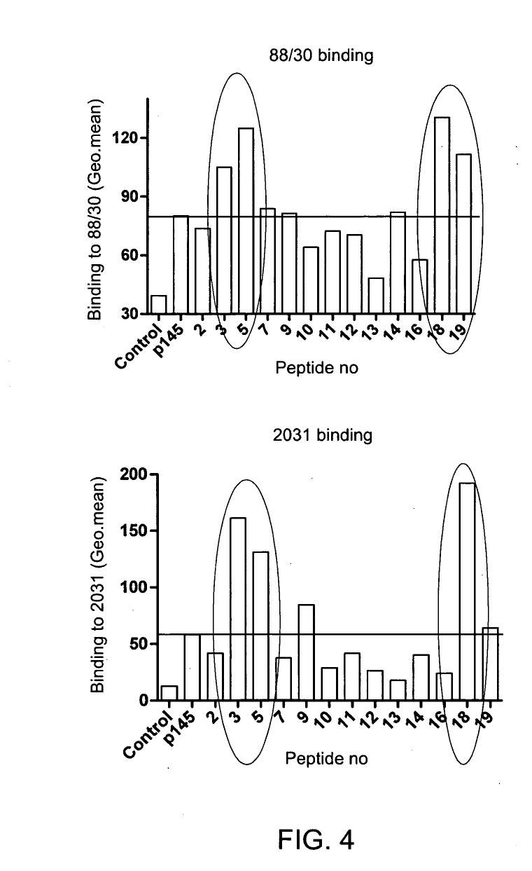

Using flow cytometry, the ability of purified TN peptide specific antibodies to bind to GAS strains 2031 and 88/30 was analysed. Anti-mouse IgG labelled with fluorescein isothiocyanate (FITC) was used to detect binding of the TN peptide specific antibodies. The results are presented in FIG. 4.

### Example 9

### Binding of Purified Anti-TN Peptide 18 Antibody to GAS

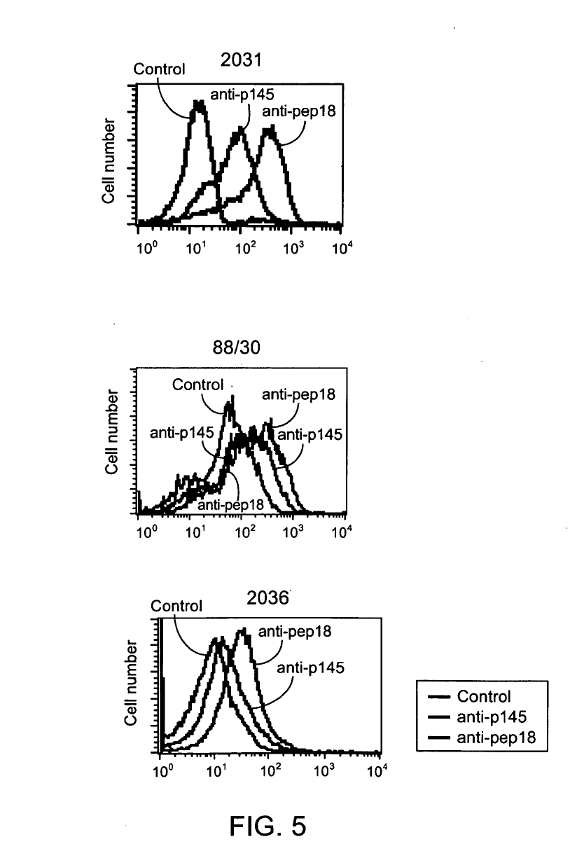

The binding of purified anti-TN peptide 18 antibodies to three strains of GAS (2031, 1036 and 88/30) was tested. The results are presented in FIG. 5. Purified anti-TN peptide 18 antibodies bind better than anti-p145 antibodies to all three GAS strains

### Example 10

### Binding of Anti-TN Peptide 18 Antibody is Dose Dependent

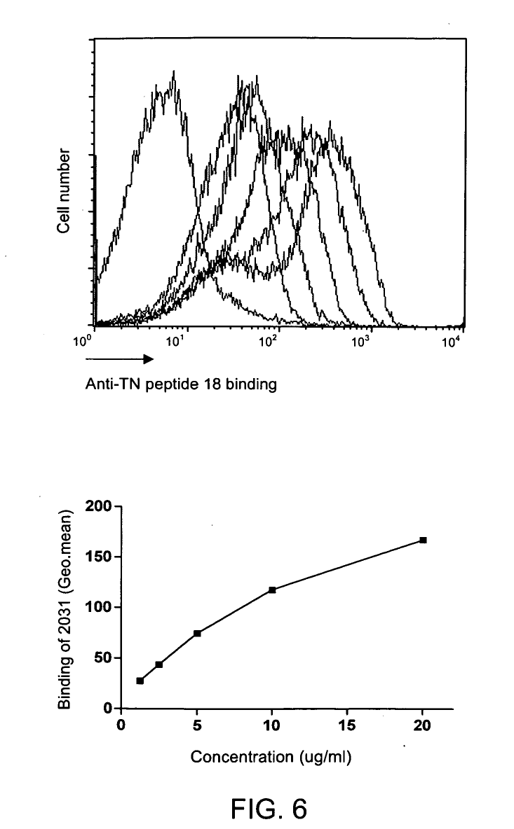

The dose dependency of anti-TN peptide 18 antibody to GAS 2031 was investigated. The results are presented in FIG. 6.

### Example 11

### Binding of Anti-TN Peptide Sera to Different p145-Derived Peptides

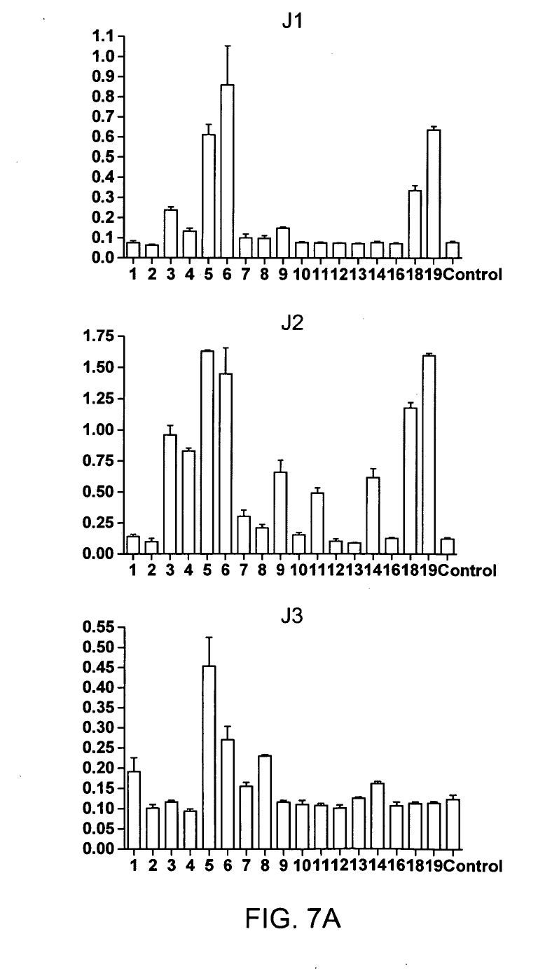

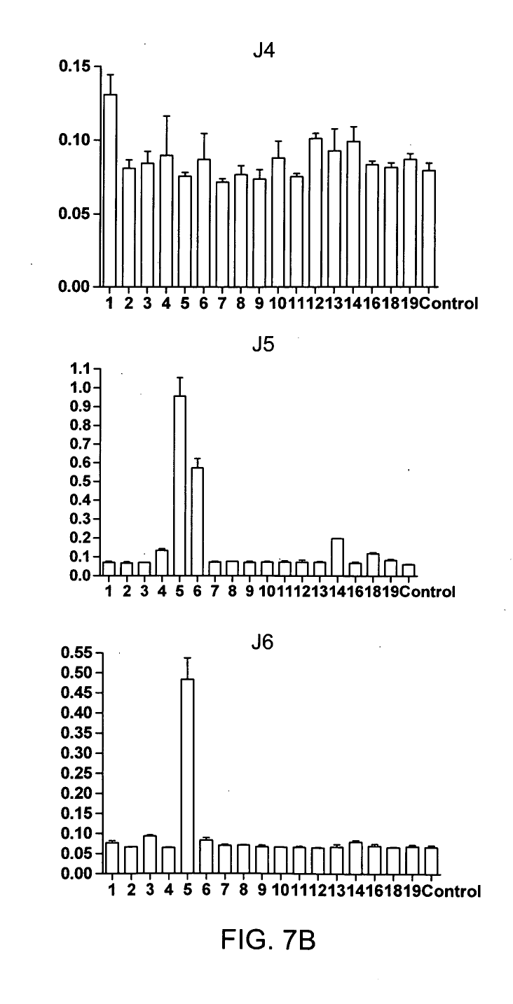

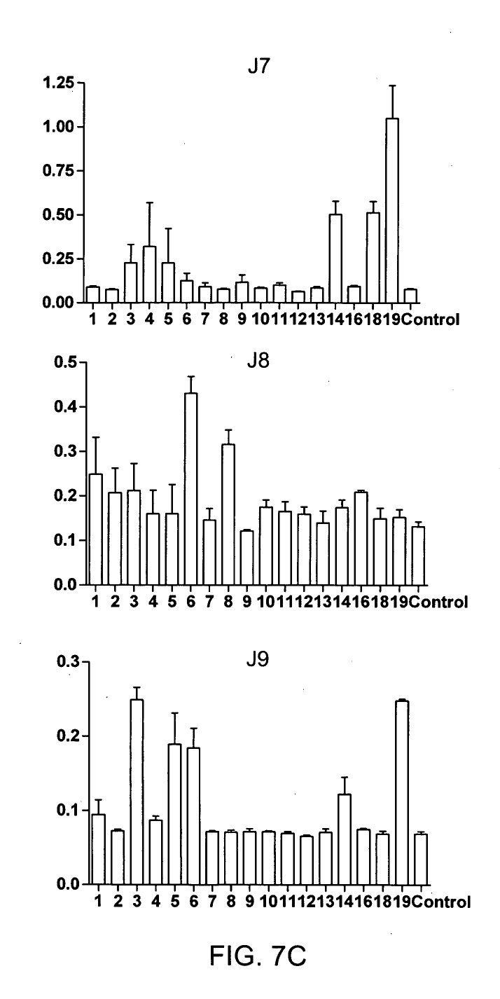

The binding of anti-TN peptide sera to the following series of peptides was investigated. The results are presented in FIGS. 7A-7C.

| | |
|---|---|
| p145 | LRRDLDASREAKKQVEKALE |
| J1 | LRRDLDASREAK |
| J2 | RRDLDASREAKK |
| J3 | RDLDASREAKKQ |
| J4 | DLDASREAKKQV |
| J5 | LDASREAKKQVE |
| J6 | DASREAKKQVEK |
| J7 | ASREAKKQVEKA |
| J8 | SREAKKQVEKAL |
| J9 | REAKKQVEKALE |
| J14 | ASREAKKQVEKALE |

J1-J9 are 12 amino acid contiguous fragments of p145, each fragment starting one amino acid on from the previous fragment. J14 is a 14 amino acid fragment of p145. The TN peptide antisera bound to J1-9 and J14 peptides as follows:

TN Peptide antisera no.

| | | |
|---|---|---|
| p145 | LRRDLDASREAKKQVEKALE | |
| J1 | LRRDLDASREAK | 5, 6, 18, 19 |
| J2 | RRDLDASREAKK | 3, 4, 5, 6, 9, 14, 18, |
| | | 19 |
| J3 | RDLDASREAKKQ | 1, 5, 6, 8, 14 |
| J4 | DLDASREAKKQV | |
| J5 | LDASREAKKQVE | 5, 6 |
| J6 | DASREAKKQVEK | 5 |
| J7 | ASREAKKQVEKA | 14, 18, 19 |
| J8 | SREAKKQVEKAL | 3, 6, 7, 8 |
| J9 | REAKKQVEKALE | 3, 5, 6, 14, 19 |
| J14 | ASREAKKQVEKALE | 5, 14, 19 |

These results indicate that anti-TN peptide 18 antisera demonstrates good overall binding in terms of titres, binding to various GAS strains and binding to multiple epitopes within the p145 sequence. Peptide TN18 differs from the p145 sequence by having (i) an asparagine in place of a lysine at position 13 of the P145 sequence, and (ii) an arginine in place of a lysine at position 17 of the p145 sequence.

It was also noted that anti-TN peptide 19 antisera bound to peptides J7, J9 and J14. This peptide and antisera thereto would appear also to represent a potential useful new vaccine/therapeutic candidate. Similarly, other peptides and antisera thereto, for example peptides TN4 and TN6, would also appear to represent potentially useful new vaccine/therapeutic candidates.

### Example 12

### Binding of J18 and J19 Peptides

Chimeric peptides designated J18 and J19, based on the TN18 and TN19 peptides, respectively, were created. The sequences of the J18 and J19 peptides compared to the p145 peptide and chimeric J8 peptide are given below. Bold underlined letters indicate amino acid positions that were changed and found to enhance immunogenicity. Underlined letters indicate the second amino acid framework peptide sequence.

| | |
|---|---|
| P145: | LRRDLDASREAKKQVEKALE |
| J8: | QAEDKVKQSREAKKQVEKALKQLEDKVQ |
| TN18: | LRRDLDASREAKNQVERALEC |
| J18: | QAEDKVKQSREAKNQVERALKQLEDKVQ |
| TN19: | LRRDLDASREAKNQVEMALEC |
| J19: | QAEDKVKQSREAKNQVEMALKQLEDKVQ |

Female B10.BR mice were immunized with TN18, TN19, J18 and J19 peptides conjugated to diphtheria toxoid (DT). The following immunization protocol was used.

| Vaccination | Sera Collection |
|---|---|
| | Day −1; Pre-vaccination 10 ul |
| Day 0; Immunization of 30 ug in CFA | |
| | Day 20; 10 ul |
| Day 21; Boost of 30 ug in PBS | |
| | Day 27; 10 ul |
| Day 28; Boost of 30 ug in PBS | |
| | Day 35; Full Bleed 200-300 ul |
| | Day 71; 10 ul |
| Day 76; Boost of 30 ug in PBS | |
| | Day 83; Bleed Out |

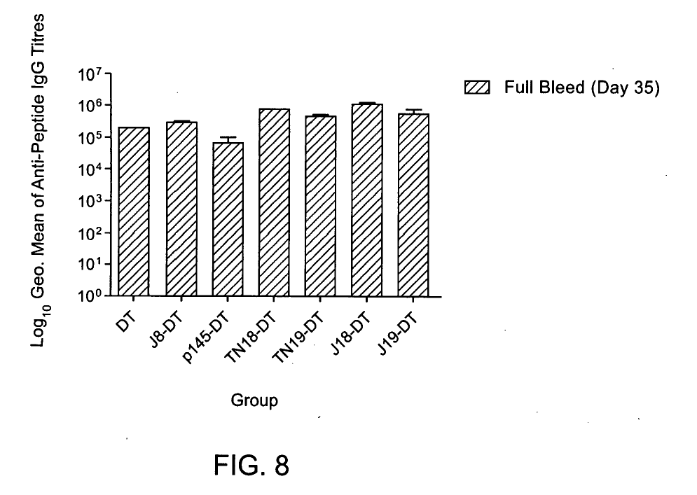

The peptide specific titres in the murine sera samples on day 35, after primary immunization and two boosters, were analyzed. The results are presented in FIG. 8.

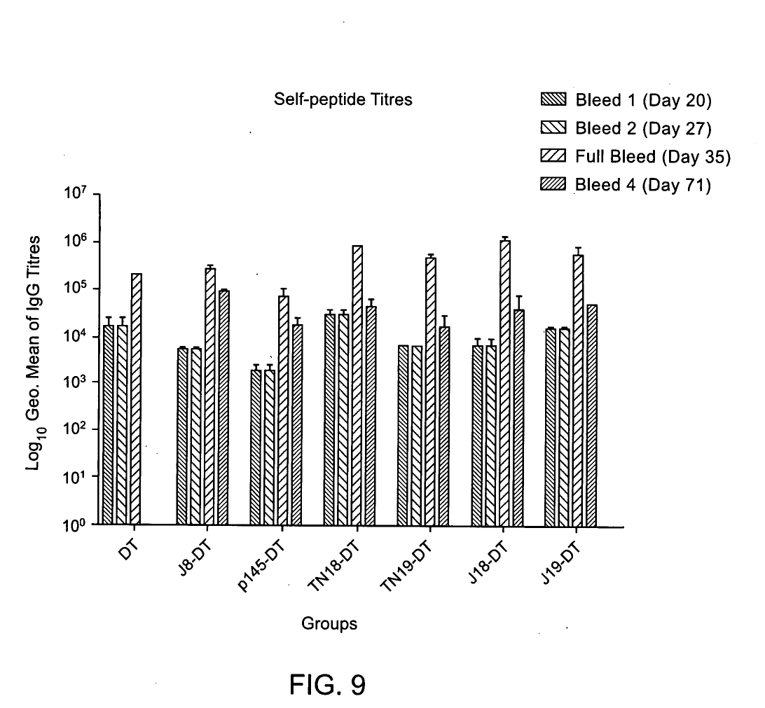

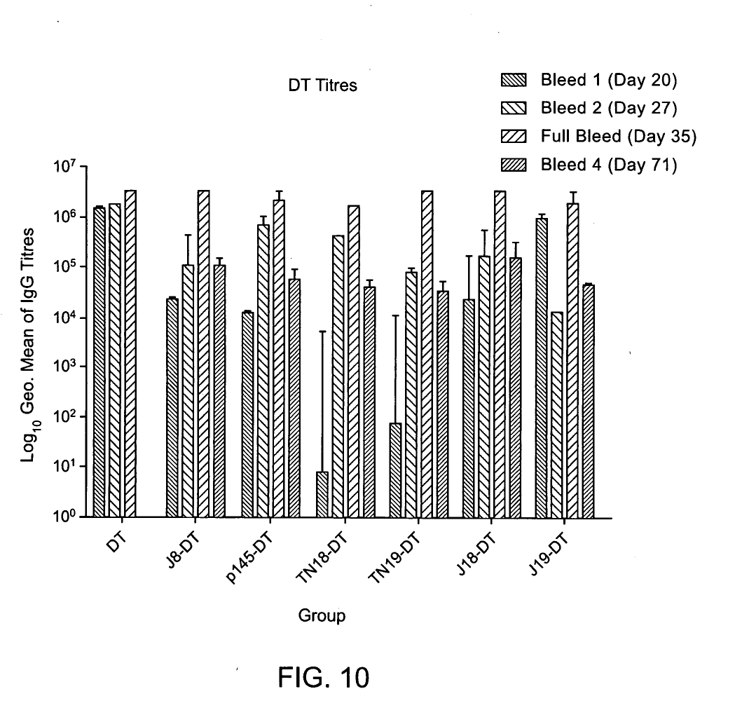

The self-peptide titres throughout the immunization protocol were analyzed at day 20, day 27, day 35 and day 71. The results are presented in FIG. 9. The diphtheria toxoid titres were also analysed throughout the immunization protocol and the results are presented in FIG. 10.

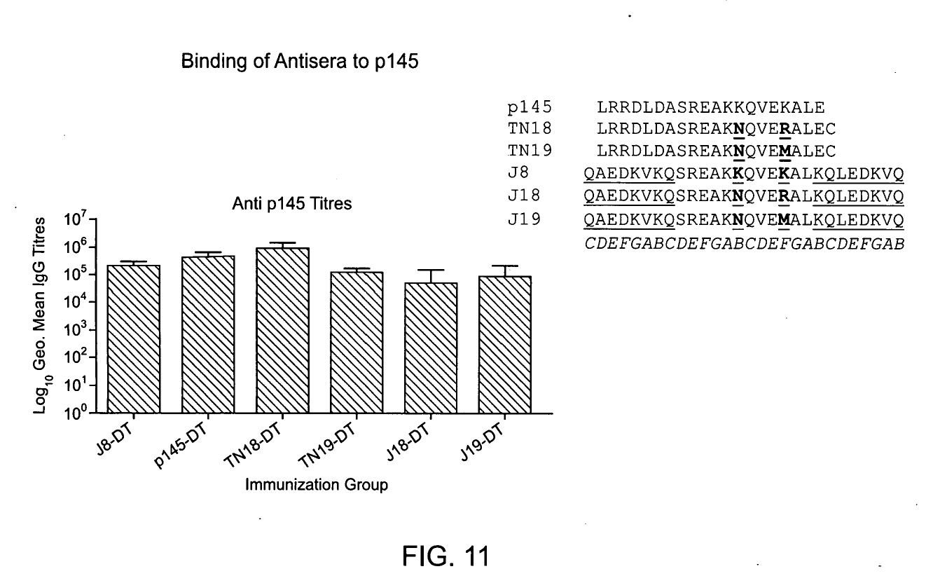

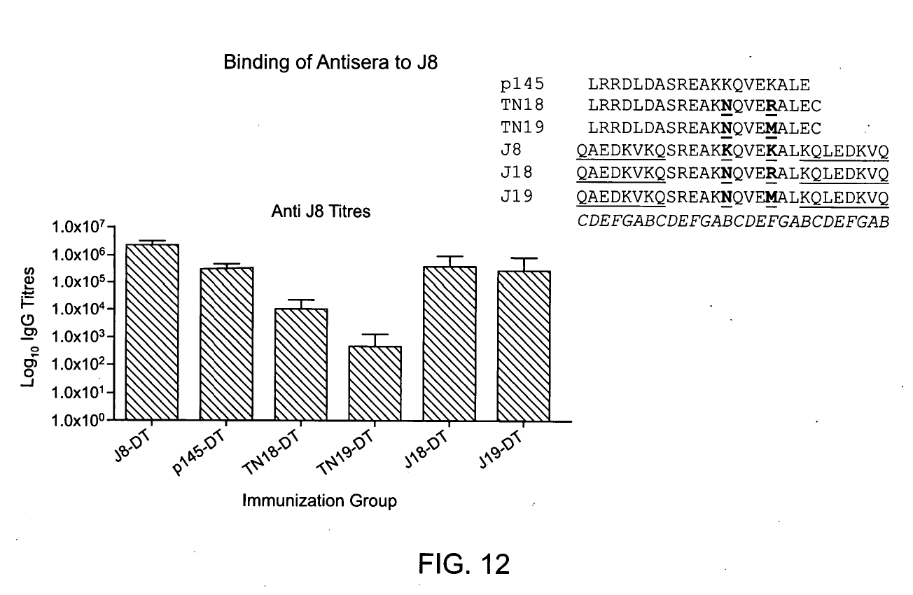

The binding of antisera to p145 and J8 were also monitored. The results are shown in FIG. 11 and FIG. 12, respectively.

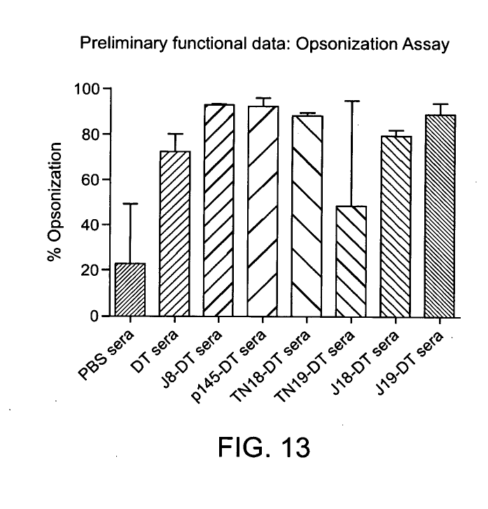

The opsonisation of 88/30 GAS by immune sera was compared tonoraml mouse sera. Preliminary functional data obtained from these opsonisation assays is shown in FIG. 13.

The examples described above show that the J18 and J19 chimeric peptides represent vaccine candidates with improved immunogenicity over the peptides disclosed in WO96/11944 against Group A Streptococcus (GAS).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of the steps or features.

### LIST OF REFERENCES

Ausubel et al. (1987). Current Protocols in Molecular Biology, Greene Publishing and Wiley-Interscience, New York.
Beachy E H, Bronze M, Dale J B, Kraus W, Poirier T and Sargent S, (1988) Vaccine 6: 192-196.
Berger & Kimmel ((1987). Methods in Enzymology, Vol. 152, Guide to Molecular Cloning Techniques, Academic Press, Inc., San Diego, Calif.
Bisno A L, et al., Clin Infect Dis 2005 41(8): 1150-1156.
Cohen C and Parry D A D (1990) Proteins: structure, functional and genetics 7: 1-15.
Cohen C and Parry D A D (1986) TIBS 11: 245-248.
Creighton (1984) Proteins, W.H. Freeman and Company
Gengyo-Ando K and Kagawa H (1991) J. Mol. Biol. 219: 429-441.
Geysen H M, Rodda S J, Mason T J, Tribbick G and Schoofs P G (1987) J. Immunological Methods 102: 259-274.
Harbury P B, Kim P S and Alber T (1994) Nature 371: 80-83.
Harbury P B, Zhang T, Kim P S and Alber T (1993) Science 262: 1401-1407.
Heyman W A, et al., Int Immunology 1997 8(11):1723-1733
Houghten R A (1985) Proc. Natl. Acad. Sci. USA 82: 5131-5135.
Liew C C et al (1990) Nucl. Acids. Res. 18: 3647.
Lupas A, van Dyke M and Stock J (1991) Science 252: 1162-1164.
Manula and Fischetti (1980) J. Exp. Med. 151: 695-708.
Merrifield. (1963). J. Am. Chem. Soc., 85, 2149-2156; Atherton et al. (1989). Solid Phase Peptide Synthesis, IRL Press, London.
Metzger et al. (1988). Nature, 334, 31-36. O'Shea E K, Rutkowski R and Kim P S (1989) Science 243: 538-542.
O'Shea E K, Klemm J D, Kim P S and Alber T (1991) Science 254: 539-544.
Pruksakorn S, Galbraith A, Houghten R A and Good M F (1992) J. Immunol. 149: 2729-2735.
Pruksakorn S, (1994) PhD thesis, University of Queensland.
Pruksakorn S, Currie B, Brandt E, Martin D, Galbraith A, Phomphutkul C, Hunsakunachai S, Manmontri A and Good M F (1994a) Lancet 344: 639-642.
Pruksakorn S, Currie B, Brandt E, Phomphutkul C, Hunsakunachai S, Manmontri A, Robinson J H, Kehoe M A, Galbraith A and Good M F (1994b) Intl. Immunol. 6: 1235-1244.
Saez, L et al (1990) Nucl. Acids. Res. 14: 2951.
Sambrook et al. ((1989). Molecular Cloning—A Laboratory Manual, 2nd ed., vols. 1-3, Cold Spring Harbor Laboratory. Cold Spring.
Scott J K and Smith G P, (1990) Science 249: 386-390.
Waterston R H et al (1977) J. Mol. Biol. 177: 679-697.
Yan Y, Winograd E, Viel A, Cronin T, Harrison S C and Branton D, (1993) Science 262: 2027-3030.

1. A chimeric peptide comprising a first amino acid sequence comprising a conformational epitope inserted within a second amino acid sequence wherein the first and second amino acid sequences are derived from peptides, polypeptides or proteins having similar native conformations, wherein the first amino acid sequence has at least three amino acids selected from within the following sequence:
(SEQ ID NO: 1)
L-R-R-D-L-D-A-X¹-X²-E-A-K-X³-Q-V-E-X⁴-A-L-E
wherein
X¹ is selected from S, E and V;
X² is selected from R, N and D;
X³ is selected from K and N; and
X⁴ is selected from K, R and M,
wherein the at least three amino acids constitute a conformational epitope and wherein at least one of the at least three amino acids is selected from the group consisting of X¹ being E or V, X² being N or D, X³ being N and X⁴ being R or M.

2. A chimeric peptide according to claim 1, wherein the first amino acid sequence has at least three amino acids selected from within the following sequence:

| | |
|---|---|
| X¹-X²-E-A-K-X³-Q-V-E-X⁴-A-L | (SEQ ID NO: 2) |

wherein
X¹ is E or V;
X² is N or D;
X³ is N; and
X⁴ is R or M.

3. A chimeric peptide according to claim 1, wherein the first amino acid sequence comprises from 10 to 15 contiguous amino acid residues of SEQ ID NO:1.

4. A chimeric peptide according to claim 1, wherein the first amino acid sequence is selected from the group consisting of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, or SEQ ID NO:20.

5. A chimeric peptide according to claim 1, wherein the first amino acid sequence consists of the sequence selected from S-R-E-A-K-N-Q-V-E-R-A-L (SEQ ID NO: 19) or S-R-E-A-K-N-Q-V-E-M-A-L (SEQ ID NO:20).

6. A chimeric peptide according to claim 1, wherein the second amino acid sequence has a similar conformation to the first amino acid sequence in its native state, and is derived from a completely unrelated protein, polypeptide or peptide.

7. A chimeric peptide according to claim 1, wherein the chimeric peptide is selected from the group consisting of SEQ ID NO:23, SEQ ID NO:24, SEQ ID NO:25, SEQ ID NO:26, SEQ ID NO:27, SEQ ID NO:28, SEQ ID NO:29, SEQ ID NO:30, or SEQ ID NO:31 wherein X⁵ is selected from K, R and M.

8. A chimeric peptide according to claim 1, wherein the second amino acid sequence has a similar conformation to the first amino acid sequence in its native state, and is derived from a related protein, polypeptide or peptide.

9. A chimeric peptide according to claim 1, wherein the chimeric peptide is selected from the group consisting of SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49.

10. A vaccine useful against streptococci, the vaccine comprising a chimeric peptide according to claim 1 and one or more pharmaceutically acceptable carriers and/or excipients.

11. An antibody, or fragment or conjugate thereof, which binds immunospecifically to a conformational epitope of a streptococcal, the conformational epitope comprising at least three amino acids from within SEQ ID NO:1 wherein X¹ is selected from S, E and V; X² is selected from R, N and D; X³ is selected from K and N; and X⁴ is selected from K, R and M, and wherein at least one amino acid of the at least three amino acids is selected from the group consisting of X¹ being E or V, X² being N or D, X³ being N and X⁴ being R or M.

12. An antibody, or fragment or conjugate thereof, which binds immunospecifically to a conformational epitope comprising at least three amino acids from within SEQ ID NO:2 wherein X¹ is selected from S, E and V; X² is selected from R, N and D; X³ is selected from K and N; and X⁴ is selected from K, R and M, and wherein at least one amino acid of the at least three amino acids is selected from the group consisting of X¹ being E or V, X² being N or D, X³ being N and X⁴ being R or M.

13. An antibody, or fragment or conjugate thereof according to claim 12, wherein the at least one amino acid of the at least three amino acids constituting the conformation epitope is selected from the group consisting of X¹ being E or V, X² being N or D, X³ being N and X⁴ being R or M.

14. An antibody, or fragment or conjugate thereof according to claim 12, wherein the at least one amino acid is selected from the group consisting of X³ being N and X⁴ being R or M.

15. An antibody, or fragment or conjugate thereof according to claim 12, wherein the conformational epitope comprises at least X³ being N and X⁴ being R or M.

16. An antibody, or fragment or conjugate thereof according to claim 12, wherein the conformational epitope comprises X¹ being S, X² being R, X³ being N and X⁴ being R or M.

17. An antibody, or fragment or conjugate thereof according to claim 12, wherein the antibody binds immunospecifically to a conformational epitope present within a sequence selected from the group consisting of SEQ ID NO:3 to SEQ ID NO:20.

18. An antibody, or fragment or conjugate thereof according to claim 12, wherein the antibody binds immunospecifically to a conformational epitope present within SEQ ID NO:19 or SEQ ID NO:20.

19. An antibody, or fragment or conjugate thereof according to claim 11, wherein the antibody is selected from the group consisting of an immunoglobulin fraction, monoclonal antibody, recombinant antibody or humanised antibody.

20. A pharmaceutical composition comprising one or more antibodies, antibody fragments or antibody conjugates according to claim 11 together with one or more pharmaceutically acceptable carriers and/or excipients.

21. A method of neutralizing a streptococcal pathogen in a subject exposed to the pathogen, comprising administering to the subject infected with the streptococcal pathogen a pharmaceutical composition according to claim 20, wherein the composition is administered in an amount effective to opsonize the pathogen in the serum of the subject.

22. A method of maintaining a therapeutically or prophylactically effective serum titre of an antibody against a streptococcal M protein in a subject, the method comprising administering a plurality of doses of a pharmaceutical composition according to claim 20, wherein each of the doses is administered in an amount effective to prevent an increase in bacterial count or to reduce bacterial count in a sample from the subject and/or to reduce the severity of one or more disease symptoms or to prevent onset of one or more diseases arising from a streptococcal, and/or to opsonize the pathogen in the serum of the subject.

23. A method of treating or ameliorating a streptococcal infection in a human or other mammalian subject, comprising administering to the subject a composition according to claim 20, wherein the composition is administered in an amount effective to prevent an increase in bacterial count or to reduce bacterial count in a sample from the subject.

24. A method for the diagnosis of streptococcal infection in a subject comprising contacting a biological sample from the subject with an antibody binding effective amount of a chimeric peptide complex to form, and then detecting the complex.
